(19) United States
(12) Patent Application Publication
(10) Pub. No.: US 2004/0158065 A1
(43) Pub. Date: Aug. 12, 2004

(54) Preparation of substituted quinazolines

(76) Inventors: Hubert Barth, Emmendingen (DE); Alexander James Bridges, Saline, MI (US); Ronald J. Heemstra, Pinckney, MI (US); Nicole Marcia Horne, Muskegon, MI (US); Robert Craig Hughes, Grand Haven, MI (US); Thomas Elliott Jacks, Holland, MI (US); Dennis Joseph McNamara, Ann Arbor, MI (US); Simon Schneider, Merzhausen (DE); Klaus Steiner, Emmendingen (DE); Peter Laurence Toogood, Ann Arbor, MI (US); Roy Thomas Winters, Pinckney, MI (US)

Correspondence Address: Craig Bell, Pfizer, Inc, 150 East 42nd Street, New York, NY, 10017-5755, US

(21) Appl. No.: 10/771,774

(22) Filed: Feb. 4, 2004

Related U.S. Application Data
(60) Provisional application No. 60/445,095, filed on Feb. 5, 2003.

(51) Int. Cl. C 07D 4 3/02

(52) U.S. Cl. 544/284; 544/293; 544/244

(57) ABSTRACT

Methods and materials for preparing irreversible inhibitors of tyrosine kinases of general Formula 1 are disclosed. Such inhibitors, which include N-[4-(3-chloro-4-floro-phenylamino)-7-(3-morpholin-4-yl-propoxy)-quinazolin-6-yl]-acrylamide, are useful for treating cancer, restenosis, atherosclerosis, endometriosis and psoriasis. The disclosed methods employ protecting schemes to minimize undesirable diacryloylamino-quinazoline side products.

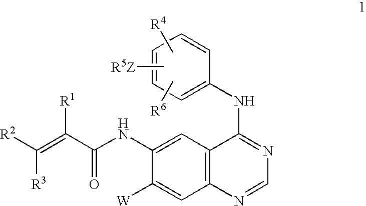

### BACKGROUND OF THE INVENTION

1. Field of Invention

This invention relates to materials and methods for preparing irreversible inhibitors of tyrosine kinases, and more particularly, to materials and methods for preparing 4,6,7-trisubstituted quinazolines, such as N-[4-(3-chloro-4-fluoro-phenylamino)-7-(3-morpholin-4-yl-propoxy)-quinazolin-6-yl]-acrylamide. These compounds are useful for treating cancer, restenosis, atherosclerosis, endometriosis, and psoriasis.

2. Discussion

Certain substituted quinazolines (see Formula 1 below) have been shown to irreversibly inhibit a family of tyrosine kinase epidermal growth factor receptors (erbB1, erbB2, erbB3 and erbB4). See commonly assigned U.S. Pat. Nos. 6,127,374, 6,153,617, 6,344,455, and 6,344,459, which are herein incorporated by reference in their entirety for all purposes. These receptors have been implicated in diseases associated with undesirable cell proliferation, including cancer, restenosis, atherosclerosis, endometriosis, and psoriasis. This suggests that irreversible inhibitors of tyrosine kinases should prove useful in treating cancer and other diseases associated with undesirable cell proliferation. Indeed, recent studies indicate that N-[4-(3-chloro-4-fluoro-phenylamino)-7-(3-morpholin-4-yl-propoxy)-quinazolin-6-yl]-acrylamide and structurally related compounds are potentially potent anti-cancer agents. See, e.g., U.S. Pat. No. 6,344,455.

One approach to making compounds of Formula 1 is based on WO 01/62743, which discloses a one-pot synthesis of (3-chloro-4-fluoro-phenyl)-[7-(3-morpholin-4-yl-propoxy)-6-aminoquinazolin-4-yl]-amine. This diamine can be reacted with a suitable acylating agent (e.g., an activated acrylic acid derivative) to yield a desired 6-acryloylamino-4-anilino-7-(oxy, sulfanyl or amino)-quinazoline. One difficulty with this approach, however, is the potential for unwanted acryloylation of the 4-anilino moiety, which would decrease yields of the desired compound and complicate the purification process. Thus, other methods are needed to prepare compounds of Formula 1.

### SUMMARY OF THE INVENTION

The present invention provides methods and materials for preparing compounds of Formula 1. The claimed methods employ protection strategies that minimize undesirable side-reaction of the anilino moiety, thereby improving yields and simplifying purification of desired products, including their pharmaceutically acceptable salts and esters. The claimed methods are particularly useful for preparing N-[4-(3-chloro-4-fluoro-phenylamino)-7-(3-morpholin-4-yl-propoxy)-quinazolin-6-yl]-acrylamide, which is an irreversible tyrosine kinase inhibitor.

Thus, one aspect of the present invention provides a method of making a compound of Formula 1,

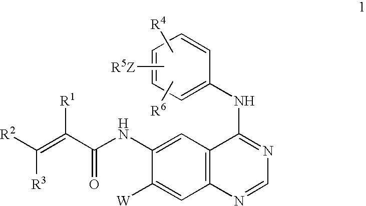

in which R¹, R² and R³ are independently hydrogen, halogen, NO₂, CN, CF₃, C₁₋₆ alkyl, C₁₋₆ haloalkyl, C₂₋₆ alkenyl, C₂₋₆ alkynyl, C₃₋₈ cycloalkyl, C₃₋₈ heterocyclyl, carboxy, C₁₋₆ alkoxycarbonyl, C₁₋₆ alkylcarbamoyl, aryl-(CH₂)ₘ, heteroaryl-(CH₂)ₘ, heterocyclyl-(CH₂)ₘ, (CH₂)ₘCO₂R⁸, (CH₂)ₘS(O)ₙR⁸, (CH₂)ₘSO₂NR⁸R⁹, OR⁸, SR⁸, (CH₂)ₘNR⁸R⁹, (CH₂)ₘN(O)R⁸R⁹, (CH₂)ₘP(O)(OR⁸)(OR⁹), (CH₂)ₘCOR⁸, (CH₂)ₘCO₂R⁸, (CH₂)ₘC(O)NR⁸R⁹, (CH₂)ₘC(O)NR⁸SO₂R⁸, (CH₂)ₘNR⁸SO₂R⁹, (CH₂)ₘC(O)NR⁸OR⁹, (CH₂)ₘS(O)ₙR⁸, or (CH₂)ₘSO₂NR⁸R⁹, wherein aryl-(CH₂)ₘ includes phenylalkyl or substituted phenylalkyl having from one to three substituents that are independently NO₂, CN, CF₃, C₁₋₆ alkyl-NH, (C₁₋₆ alkyl)₂N, or monocyclic heteroaryl, and each C₁₋₆ alkyl is optionally substituted with OH, NH₂ or —N(A)B;

R⁴ and R⁶ are independently hydrogen, hydroxy, halogen, C₁₋₄ alkyl, C₁₋₄ alkoxy, C₁₋₄ alkylamino, C₁₋₄ alkyldiamino, C₁₋₄ alkylthio, C₁₋₄ alkylsulfinyl, C₁₋₄ alkylsulfonyl, C₁₋₄ alkylcarbonyl, C₁₋₄ alkylcarbamoyl, dicarbamoyl, carbamyl, C₁₋₄ alkoxycarbonyl, cyano, nitro, or trifluoromethyl;

R⁵ is phenyl, pyridyl, furyl, thiazolyl, imidazolyl or thienyl, each optionally having one or two substituents that are independently halogen, C₁₋₆ alkyl, C₁₋₆ alkoxy, hydroxy, amino, cyano, C₁₋₆ alkyl-NH or (C₁₋₆ alkyl)₂N;

W is SR⁷, OR⁷ or NHR⁷; and

Z is hydrogen, halogen, C₁₋₆ alkyl, C₃₋₈ cycloalkyl, C₁₋₆ alkoxy, C₃₋₈ cycloalkoxy, nitro, C₁₋₆ haloalkyl, hydroxy, C₁₋₆ acyloxy, NH₂, C₁₋₆ alkyl-NH, (C₁₋₆ alkyl)₂N, C₃₋₈ cycloalkyl-NH, (C₃₋₈ cycloalkyl)₂N, hydroxymethyl, C₁₋₆ alkylcarbonyl, cyano, azido, C₁₋₆ thioalkyl, C₁₋₆ sulfinylalkyl, C₁₋₆ sulfonylalkyl, C₃₋₈ thiocycloalkyl, C₃₋₈ sulfinylcycloalkyl, C₃₋₈ sulfonylcycloalkyl, mercapto, C₁₋₆ alkoxycarbonyl, C₃₋₈ cycloalkoxycarbonyl, C₂₋₄ alkenyl, C₄₋₈ cycloalkenyl, or C₂₋₄ alkynyl, provided that when Z is monovalent, R⁵ is absent;

wherein, R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;

R⁸ and R⁹ are each independently hydrogen, C₁₋₆ alkyl, C₁₋₆ haloalkyl, C₂₋₆ alkenyl, C₂₋₆ alkynyl, arylalkyl, cycloalkyl, heterocyclyl, aryl, heteroaryl, or heteroarylalkyl;

A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and

n and m are, respectively, integers from zero to two, inclusive, and from zero to four, inclusive.

The method includes removing a protecting group, G, from a compound of Formula 10,

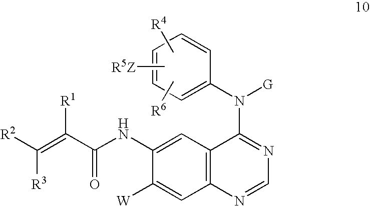

to yield the compound of Formula 1, and optionally converting the compound of Formula 1 to a pharmaceutically acceptable salt, ester, amide, or prodrug.

Another aspect of the present invention provides a method of making a compound of Formula 23,

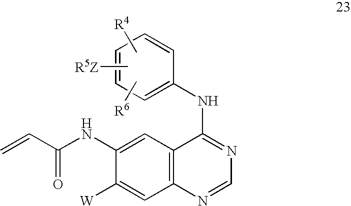

where R⁴, R⁵, R⁶, W and Z are as defined above for Formula 1. The method includes eliminating SR¹² from a compound of Formula 22,

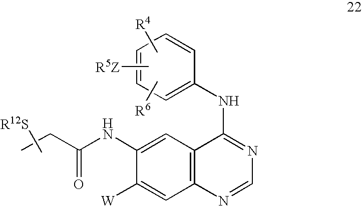

to yield the compound of Formula 23, and optionally converting the compound of Formula 23 to a pharmaceutically acceptable salt, ester, amide, or prodrug. In Formula 22, R¹²S is bonded to the 2- or 3-position carbon atom of the propionamido group, and substituent R¹² is C₁₋₆ alkyl or aryl.

A further aspect of the present invention provides a method of making a compound of Formula 29,

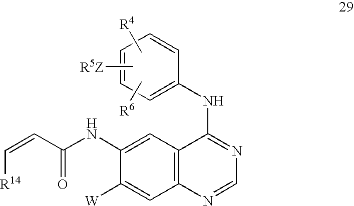

where R⁴, R⁵, R⁶, W and Z are as defined above for Formula 1 and R¹⁴ is hydrogen, halogen, C₂₋₆ alkenyl, C₂₋₆ alkynyl, and C₂₋₆ alkenyl or C₂₋₆ alkynyl substituted with hydroxy, alkoxy, amino or alkylamino. The method includes removing [1,3,4]oxadiazole from a compound of Formula 28,

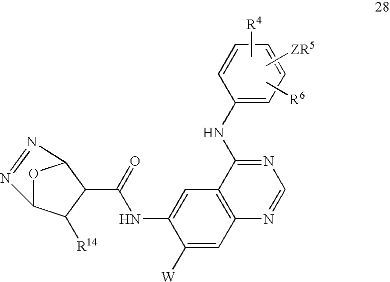

to yield the compound of Formula 29, and optionally converting the compound of Formula 29 to a pharmaceutically acceptable salt, ester, amide, or prodrug.

An additional aspect of the present invention provides a method of making a compound of Formula 46,

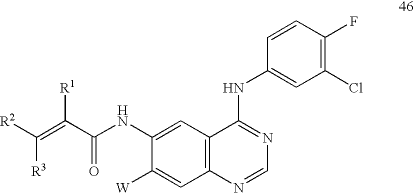

where R¹, R², R³ and W are as defined above for Formula 1. The method includes treating a compound of Formula 45,

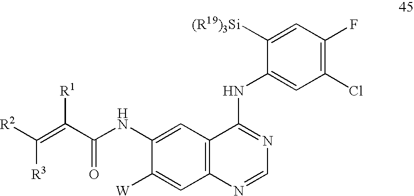

with an acid to yield the compound of Formula 46, wherein R¹⁹ is C₁₋₄ alkyl, C₁₋₄ alkoxy, or aryl, and optionally converting the compound of Formula 46 to a pharmaceutically acceptable salt, ester, amide, or prodrug.

Another aspect of the present invention provides compounds of Formula 47,

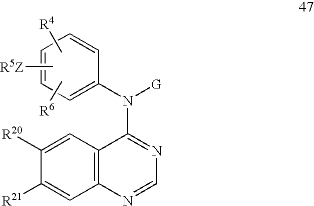

or pharmaceutically acceptable salts, esters, amides or prodrugs thereof, in which R²⁰ is NH₂, NO₂, or

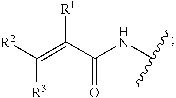

R²¹ is SR⁷, OR⁷, NHR⁷ or a leaving group; R¹, R², R³, R⁴, R⁵, R⁶, R⁷, and Z are as defined in Formula 1; and G is as defined above in Formula 10, provided that when G is Boc and R²⁰ is NH₂ or NO₂, R²¹ is not halogen or alkoxy.

Another aspect of the present invention provides one or more compounds (or their pharmaceutically acceptable salts) selected from:

(3-chloro-4-fluoro-phenyl)-(3,4-dimethoxy-benzyl)-(7-fluoro-6-nitro-quinazolin-4-yl)-amine;

(3-chloro-4-fluoro-phenyl)-(3,4-dimethoxy-benzyl)-[7-(3-morpholin-4-yl-propoxy)-6-nitro-quinazolin-4-yl]-amine;

N4-(3-chloro-4-fluoro-phenyl)-N4-(3,4-dimethoxy-benzyl)-7-(3-morpholin-4-yl-propoxy)-quinazoline4,6-diamine;

N-[4-[(3-chloro-4-fluoro-phenyl)-(3,4-dimethoxy-benzyl)-amino]-7-(3-morpholin-4-yl-propoxy)-quinazolin-6-yl]-acrylamide;

N-(3-chloro-4-fluoro-phenyl)-N-[7-(3-morpholin-4-yl-propoxy)-6-nitro-quinazolin-4-yl]-acetamide;

N-[6-amino-7-(3-morpholin-4-yl-propoxy)-quinazolin-4-yl]-N-(3-chloro-4-fluoro-phenyl)-acetamide; and

N-[4-[acetyl-(3-chloro-4-fluoro-phenyl)-amino]-7-(3-morpholin-4-yl-propoxy)-quinazolin-6-yl]-acrylamide.

Another aspect of the present invention provides a compound of Formula 48,

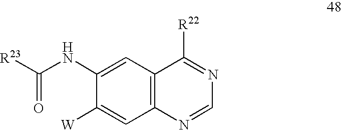

or a pharmaceutically acceptable salt, ester, amide or prodrug thereof, in which R²² is a leaving group or

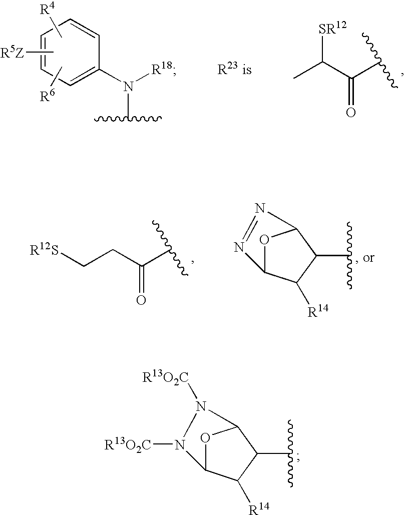

R⁴, R⁵, R⁶, W, and Z are as defined above in Formula 1; R¹² is as defined in Formula 22; R¹³ as defined in Formula 27; R¹⁴ is as defined in Formula 29; and R¹⁸ is as defined in Formula 38. Particularly useful compounds of Formula 48 include those in which R²² is

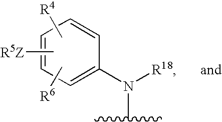

R¹⁸ is hydrogen.

Another aspect of the present invention provides a compound of Formula 49,

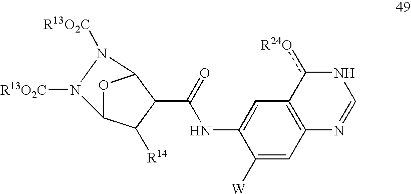

or a pharmaceutically acceptable salt, ester, amide or prodrug thereof, in which W is as defined in Formula 1; R¹³ as defined in Formula 27; R¹⁴ is as defined in Formula 29; R¹⁶ is as defined in Formula 36; and R²⁴ is P⁺(R¹⁶)₃ or is absent.

Another aspect of the present invention provides a compound of Formula 45,

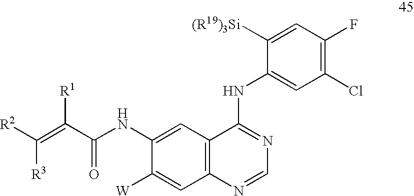

or a pharmaceutically acceptable salt, ester, amide or prodrug thereof, in which R¹, R², R³, and W are as defined above in Formula 1, and R¹⁹ is C₁₋₄ alkyl, C₁₋₄ alkoxy or aryl.

### DETAILED DESCRIPTION

Definitions and Abbreviations

Unless otherwise indicated, this disclosure uses definitions provided below. Some of the definitions and formulae may include a “—” (dash) between groups to indicate a bond. In other definitions and formulae, the dash may be omitted when it is clear that a bond exists between groups.

“Alkyl” refers to straight chain and branched aliphatic hydrocarbon groups, generally having a specified number of carbon atoms (i.e., C₁₋₆ alkyl refers to an alkyl group having from 1 to 6 carbon atoms, inclusive). Examples of alkyl groups include, without limitation, methyl, ethyl, n-propyl, i-propyl, n-butyl, s-butyl, t-butyl, n-pentyl, s-pentyl, n-hexyl, and the like.

“Alkenyl” refers to branched or unbranched hydrocarbon groups, generally having a specified number of carbon atoms, and having one or more unsaturated carbon-carbon bonds. Examples of alkenyl groups include, without limitation, ethenyl and propenyl.

“Alkynyl” refers to branched or unbranched hydrocarbon groups, generally having a specified number of carbon atoms, and having one or more triple carbon-carbon bonds. Examples of alkynyl groups include, without limitation, ethynyl and propynyl.

“Cycloalkyl” refers to saturated hydrocarbon rings, generally having a specified number of carbon atoms. Examples of cycloalkyl groups include, without limitation, cyclopropyl, cyclobutyl, cyclopentyl, cyclohexyl, and the like.

“Aminoalkyl,” “alkylamino,” “alkylaminoalkyl,” and “dialkylaminoalkyl” refer, respectively, to H₂N-alkyl, alkyl-NH, alkyl-NH-alkyl, and (alkyl)₂N-alkyl, where alkyl is defined above.

“Thioalkyl,” “thiocycloalkyl,” “alkylthio,” “alkylsulfinyl,” “sulfinylalkyl,” sulfinylcycloalkyl,” “alkylsulfonyl,” “sulfonylalkyl,” and “sulfonylcycloalkyl” refer, respectively, to HS-alkyl, HS-cycloalkyl, alkyl-S, alkyl-S(O), S(O)-alkyl, S(O)-cycloalkyl, alkyl-SO₂, SO₂-alkyl, and SO₂-cycloalkyl, where alkyl and cycloalkyl are defined above.

“Alkylcarbonyl” and “alkylcarbamoyl” refer, respectively, to alkyl-C(O) and alkyl-C(O)—NH, where alkyl is defined above.

“Alkoxy,” “thioalkoxy,” “alkoxycarbonyl,” “acyloxy,” “cycloalkoxy,” and “cycloalkoxycarbonyl” refer, respectively, to alkyl-O, alkyl-S, alkyl-O—C(O), C(O)—O, cycloalkyl-C(O), and cycloalkyl-O—C(O), where alkyl and cycloalkyl are defined above. Examples of alkoxy groups include, without limitation, methoxy, ethoxy, n-propoxy, i-propoxy, n-butoxy, s-butoxy, t-butoxy, n-pentoxy, and s-pentoxy.

“Halo,” “halogen” and “halogeno” may be used interchangeably, and refer to fluoro, chloro, bromo, and iodo.

“Haloalkyl” refers to an alkyl substituted with one or more halogen atoms, where alkyl is defined above. Examples of haloalkyl groups include, without limitation, trifluoromethyl, trichloromethyl, pentafluoroethyl, and pentachloroethyl.

“Cycloalkylalkyl” refers to a cycloalkyl group attached to an alkyl group, where cycloalkyl and alkyl are defined above. Examples of cycloalkylalkyl groups include, without limitation, cyclopropylmethyl, cyclopentylmethyl, cyclohexylmethyl, adamantylmethyl, and the like.

“Aryl” refers to monocyclic or polycyclic rings that are aromatic. Examples of aryl groups include, without limitation, phenyl, naphthyl, biphenyl, pyrenyl, anthracenyl, fluorenyl, and the like. Aryl groups may be optionally substituted with one or more substituents, such as alkyl, alkoxy, thioalkoxy, alkylcarbamoyl, alkoxycarbonyl, and alkylcarbonyl, as defined above, and hydroxy, thiol, nitro, halogen, and amino. In some aryl groups, a substituent may bridge ring atoms. Such substituents include O—(CH₂)_{q}, where q is an integer from 1 to 3.

“Arylalkyl” refers to an aryl group attached to an alkyl group, where aryl and alkyl are defined above. Examples include, without limitation, benzyl, fluorenylmethyl, and the like.

“Aryloxy” refers to an aryl-O group, where aryl is defined above.

“Heterocycle” and “heterocyclyl” refer to 5- to 7-membered monocyclic or bicyclic rings or to 7- to 10-membered bicyclic rings, which are saturated, partially unsaturated, or unsaturated. These groups have ring members made up of carbon atoms and from 1 to 4 heteroatoms that are independently nitrogen, oxygen or sulfur, and may include any bicyclic group in which any of the above-defined heterocycles are fused to a benzene ring. The nitrogen and sulfur heteroatoms may optionally be oxidized. The heterocyclic ring may be attached to a parent group or substrate at any heteroatom or carbon atom, unless such attachment would violate valence requirements. Likewise, the heterocyclyl groups may be substituted on a carbon or on a nitrogen atom, unless such substitution would violate valence requirements. Useful substituents include, but are not limited to, alkyl, alkoxy, thioalkoxy, alkylcarbamoyl, alkoxycarbonyl, and alkylcarbonyl, as defined above, and hydroxy, thiol, nitro, halogen, and amino. In some heterocyclyl groups, a substituent may bridge ring atoms. Such substituents include O—(CH₂)_{q}, where q is an integer from 1 to 3.

Examples of heterocycles include, without limitation, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Heteroaryl” refers to heterocycles or heterocyclyl groups, as defined above, which are also aromatic (i.e., aryl groups, as defined above).

“Heteroaryloxy,” “aryloyl,” and “heteroaryloyl” refer, respectively, to heteroaryl-O, aryl-C(O), and heteroaryl-C(O), where aryl and heteroaryl are defined above.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts), sulfonate esters (including tosylates, brosylates and mesylates), triflate esters, halide ions, carboxylate anions, phenolate ions, and alkoxides. Some stronger bases, such as NH₂⁻ and OH⁻ can be made better leaving groups by treatment with an acid. Common electrofugal leaving groups include the proton, CO₂, and metals.

“Pharmaceutically acceptable salts, esters, amides, or prodrugs” refer to acid or base addition salts, esters, amides, zwitterionic forms, where possible, and prodrugs of claimed and disclosed compounds, which are within the scope of sound medical judgment, suitable for use in contact with the tissues of patients without undue toxicity, irritation, allergic response, and the like, commensurate with a reasonable benefit/risk ratio, and effective for their intended use.

Examples of pharmaceutically acceptable, non-toxic esters include, without limitation, C₁₋₆ alkyl esters, C₅₋₇ cycloalkyl esters, and arylalkyl esters of claimed and disclosed compounds, where alkyl, cycloalkyl, and aryl are defined above. Such esters may be prepared by conventional methods, as described, for example, in M. B. Smith and J. March, March's Advanced Organic Chemistry (5^{th} Ed. 2001).

Examples of pharmaceutically acceptable, non-toxic amides include, without limitation, those derived from ammonia, primary C₁₋₆ alkyl amines, and secondary C₁₋₆ dialkyl or heterocyclyl amines of claimed and disclosed compounds, where alkyl and heterocyclyl are defined above. Such amides may be prepared by conventional methods, as described, for example, in March's Advanced Organic Chemistry.

“Prodrugs” refer to compounds having little or no pharmacological activity that can, when metabolized in vivo, undergo conversion to claimed or disclosed compounds having desired activity. For a discussion of prodrugs, see T. Higuchi and V. Stella, “Pro-drugs as Novel Delivery Systems,” ACS Symposium Series 14 (1975), E. B. Roche (ed.), Bioreversible Carriers in Drug Design (1987), and H. Bundgaar, Design of Prodrugs (1985).

“Treating” refers to reversing, alleviating, inhibiting the progress of, or preventing a disorder or condition to which such term applies, or to preventing one or more symptoms of such disorder or condition.

“Treatment” refers to the act of “treating,” as defined immediately above.

Table I lists abbreviations used through the specification.

TABLE I

| Abbreviation | Description |
|---|---|
| Ac | acetyl |
| AcOH | acetic acid |
| Aq | aqueous |
| Bn | benzyl |
| Boc | t-butoxycarbonyl |
| Boc-ON | 2-(Boc-oxyimino)-2-phenylacetonitrile |
| BOP | benzotriazol-1-yloxy-tris-(dimethylamino)-phosphonium |
| | hexafluorophosphate |
| Bu | butyl |
| t-BuOCl | tertiary butylhypochlorite |
| t-BuOK | potassium tertiary butyl oxide |
| t-BuOH | tertiary butyl alcohol |
| Cbz | benzyloxycarbonyl |
| DABCO | 1,4-diazabicyclo[2.2.2]octane |
| DADAOB | diacyloxydiazaoxabicycloheptane |
| DCC | dicycohexylcarbodiimide |
| DBN | 1,5-diazabicyclo[4.3.0]non-5-ene |
| DBU | 1,8-diazabicyclo[5.4.0]undec-7-ene |
| DEAD | diethylazodicarboxylate |
| DMAP | 4-dimethylaminopyridine |
| DME | dimethylether |
| DMF | dimethylformamide |
| Et | ethyl |
| EtOAc | ethyl acetate |
| EtOH | ethyl alcohol |
| FDPP | pentafluorophenyl diphenylphosphinate |
| HOAc | acetic acid |
| HOAt | 1-hydroxy-7-azabenzotriazole |
| HOBt | N-hydroxybenzotriazole |
| HODhbt | 3-hydroxy-3,4-dihydro-4-oxo-1,2,3-benzotriazine |
| Me | methyl |
| MeOH | methanol |
| MEM | methoxyethoxymethyl |
| MTBE | t-butyl methyl ether |
| NMP | N-methylpyrrolidone |
| PGMME | propylene glycol monomethyl ether |
| Ph | phenyl |
| Pr | propyl |
| i-Pr | isopropyl |
| PyBOP | benzotriazole-1-yl-oxy-tris-pyrrolidino-phosphonium |
| | hexafluorophosphate |
| TATU | 0-(7-azabenzotriazol-1-yl)-1,1,3,3-tetramethyluronium |
| | tetrafluoroborate |
| Tf | trifluoromethanesulfonyl |
| TEGMME | triethylene glycol monomethyl ether |
| TFA | trifluoroacetic acid |
| TFAA | trifluoroacetic anhydride |
| THF | tetrahydrofuran |
| TMS | trimethylsilyl |

In some of the reaction schemes and examples below, certain compounds may be prepared using protecting groups, which prevent undesirable chemical reaction at otherwise reactive sites. Protecting groups may also be used to enhance solubility or otherwise modify physical properties of a compound. For a discussion of protecting group strategies, materials and methods for installing and removing protecting groups, and a compilation of useful protecting groups for common functional groups, including amines, carboxylic acids, alcohols, ketones, aldehydes, and the like, see T. W. Greene and P. G. Wuts, Protecting Groups in Organic Chemistry (1999), and P. Kocienski, Protective Groups (2000), which are herein incorporated by reference in their entirety for all purposes.

In addition, some of the schemes and examples below may omit details of common reactions, including oxidations, reductions, and so on, which are known to persons of ordinary skill in the art of organic chemistry. The details of such reactions can be found in a number of treatises, including Richard Larock, Comprehensive Organic Transformations (1999), and the multi-volume series edited by Michael B. Smith and others, Compendium of Organic Synthetic Methods (1974-2003). Generally, starting materials and reagents may be obtained from commercial sources.

The present invention provides materials and methods for preparing compounds represented by Formula 1, including pharmaceutically acceptable salts and esters:

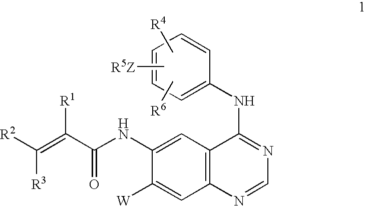

in which R¹, R² and R³ are independently hydrogen, halogen, NO₂, CN, CF₃, C₁₋₆ alkyl, C₁₋₆ haloalkyl, C₂₋₆ alkenyl, C₂₋₆ alkynyl, C₃₋₈ cycloalkyl, C₃₋₈ heterocyclyl, carboxy, C₁₋₆ alkoxycarbonyl, C₁₋₆ alkylcarbamoyl, aryl-(CH₂)ₘ, heteroaryl-(CH₂)ₘ, heterocyclyl-(CH₂)ₘ, (CH₂)ₘCO₂R⁸, (CH₂)ₘS(O)ₙR⁸, (CH₂)ₘSO₂NR⁸R⁹, OR⁸, SR⁸, (CH₂)ₘNR⁸R⁹, (CH₂)ₘN(O)R⁸R⁹, (CH₂)ₘP(O)(OR⁸)(OR⁹), (CH₂)ₘCOR⁸, (CH₂)ₘCO₂R⁸, (CH₂)ₘC(O)NR⁸R⁹, (CH₂)ₘC(O)NR⁸SO₂R⁸, (CH₂)ₘNR⁸SO₂R⁹, (CH₂)ₘC(O)NR⁸OR⁹, (CH₂)ₘS(O)ₙR⁸, or (CH₂)ₘSO₂NR⁸R⁹, wherein aryl-(CH₂)ₘ includes phenylalkyl or substituted phenylalkyl having from one to three substituents that are independently NO₂, CN, CF₃, C₁₋₆ alkyl-NH, (C₁₋₆ alkyl)₂N, or monocyclic heteroaryl, and each C₁₋₆ alkyl is optionally substituted with OH, NH₂ or —N(A)B;

R⁴ and R⁶ are independently hydrogen, hydroxy, halogen, C₁₋₄ alkyl, C₁₋₄ alkoxy, C₁₋₄ alkylamino, C₁₋₄ alkyldiamino, C₁₋₄ alkylthio, C₁₋₄ alkylsulfinyl, C₁₋₄ alkylsulfonyl, C₁₋₄ alkylcarbonyl, C₁₋₄ alkylcarbamoyl, dicarbamoyl, carbamyl, C₁₋₄ alkoxycarbonyl, cyano, nitro, or trifluoromethyl;

R⁵ is phenyl, pyridyl, furyl, thiazolyl, imidazolyl or thienyl, each optionally having one or two substituents that are independently halogen, C₁₋₆ alkyl, C₁₋₆ alkoxy, hydroxy, amino, cyano, C₁₋₆ alkyl-NH or (C₁₋₆ alkyl)₂N;

W is SR⁷, OR⁷ or NHR⁷; and

Z is hydrogen, halogen, C₁₋₆ alkyl, C₃₋₈ cycloalkyl, C₁₋₆ alkoxy, C₃₋₈ cycloalkoxy, nitro, C₁₋₆ haloalkyl, hydroxy, C₁₋₆ acyloxy, NH₂, C₁₋₆ alkyl-NH, (C₁₋₆ alkyl)₂N, C₃₋₉ cycloalkyl-NH, (C₃₋₈ cycloalkyl)₂N, hydroxymethyl, C₁₋₆ alkylcarbonyl, cyano, azido, C₁₋₆ thioalkyl, C₁₋₆ sulfinylalkyl, C₁₋₆ sulfonylalkyl, C₃₋₈ thiocycloalkyl, C₃₋₉ sulfinylcycloalkyl, C₃₋₈ sulfonylcycloalkyl, mercapto, C₁₋₆ alkoxycarbonyl, C₃₋₈ cycloalkoxycarbonyl, C₂₋₄ alkenyl, C₄₋₈ cycloalkenyl, or C₂₋₄ alkynyl, provided that when Z is monovalent, R⁵ is absent;

wherein, R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;

R⁸ and R⁹ are each independently hydrogen, C₁₋₆ alkyl, C₁₋₆ haloalkyl, C₂₋₆ alkenyl, C₂₋₆ alkynyl, arylalkyl, cycloalkyl, heterocyclyl, aryl, heteroaryl, or heteroarylalkyl;

A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and

n and m are, respectively, integers from zero to two, inclusive, and from zero to four, inclusive.

In Formula 1, representative heterocyclyl-(CH₂)ₘ substituents include piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, morpholin-4-yl-(CH₂)ₘ, thiomorpholin-4-yl-(CH₂)ₘ, hexahydroazepin-1-yl-(CH₂)ₘ. Representative heteroaryl-(CH₂)ₘ, (CH₂)ₘNR⁸R⁹, and OR⁸ substituents include, respectively, pyridinyl-(CH₂)ₘ, imidazolyl-(CH₂)ₘ, imidazol-1-yl-(CH₂)ₘ, and (CH₂)ₘNH₂, (CH₂)ₘNH(C₁₋₆ alkyl), (CH₂)ₘN(C₁₋₆ alkyl)₂, and C₁₋₆ alkoxy.

Particularly useful compounds represented by Formula 1 include those in which R¹, R² and R³ are each hydrogen, or those in which R⁴ and R⁶ are each halogen and Z is hydrogen, or those in which R¹, R², R³ and Z are each hydrogen and R⁴ and R⁶ are each halogen. Other useful compounds represented by Formula 1 include those in which W is morpholin-4-yl-alkoxy, including 3-(morpholin-4-yl)-propyloxy, or those in which R¹, R², R³ and Z are each hydrogen, R⁴ and R⁶ are each halogen, and W is a morpholin-4-yl-alkoxy. As discussed above, an especially useful compound represented by Formula 1 is an irreversible pan-erbB inhibitor, N-[4-(3-chloro-4-fluoro-phenylamino)-7-(3-morpholin-4-yl-propoxy)-quinazolin-6-yl]-acrylamide.

Scheme I illustrates a method for preparing compounds of Formula 1. The method includes providing a quinazoline starting material (Formula 2) having 4- and 7-position substituents, X¹ and X², respectively, which can be displaced by nucleophiles. Depending on the nature of the nucleophile, the leaving groups X¹ and X² are independently halogen, alkyl-O, aryl-O, acyl-O, sulfonate ester (including tosylates, brosylates, mesylates and triflate esters), carboxylate, (alkyl-O)₂P(O)O, (O-aryl)₂P(O)O, etc. The quinazoline starting material may be prepared in accordance with Scheme III, which is described below. An especially useful quinazoline starting material is 4-chloro-7-fluoro-6-nitro-quinazoline.

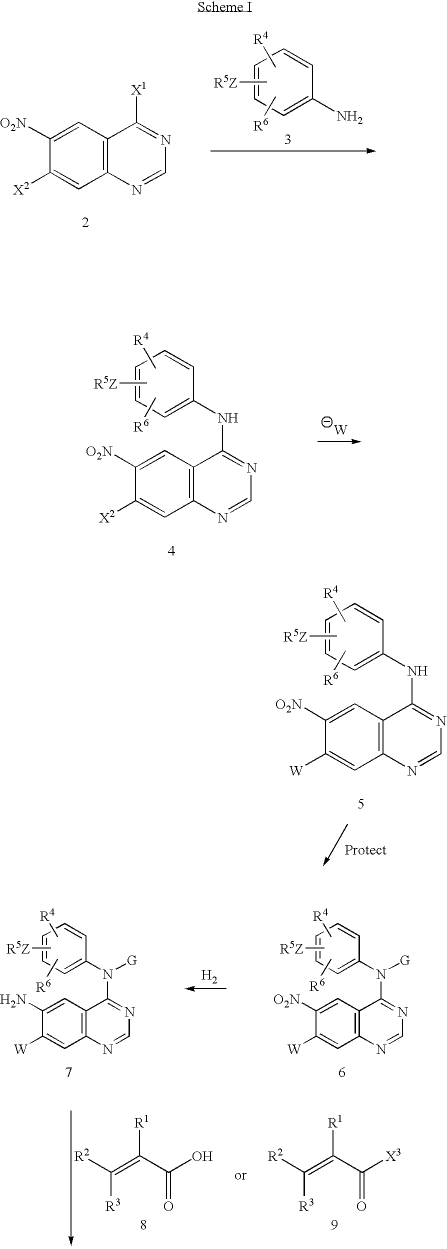

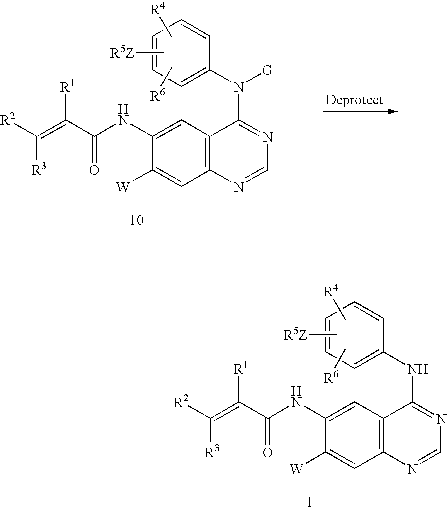

As depicted in Scheme I, the quinazoline starting material of Formula 2 is reacted with an appropriate amine (Formula 3) to produce a 4-anilino-6-nitro-quinazoline (Formula 4), which is subsequently reacted with an alcohol (R⁷OH), a thiol (R⁷SH), or a primary amine (R⁷NH₂) to yield a 4-anilino-6-nitro-quinazoline (Formula 5) having a 7-oxy, sulfanyl or amino-side chain (W). The displacement of X² typically entails deprotonation of the requisite alcohol, thiol or amine using a strong base. Suitable bases include, without limitation, potassium t-butoxide, sodium metal, sodium hydride, potassium hydride, calcium hydride, lithium bis(trimethylsilyl)amide, sodium bis(trimethylsilyl)amide, potassium bis(trimethylsilyl)amide, etc.

Note that substituents R⁴, R⁵, R⁶, W and Z in Formula 3—Formula 5 are the same as the corresponding substituents in Formula 1 (i.e., R⁴ in Formula 3—Formula 5 refers to the same substituent as R⁴ in Formula 1). More generally, and unless stated otherwise, when a particular substituent identifier (R¹, R², R³, etc.) is defined for the first time in connection with a formula, the same substituent identifier used in a subsequent formula will have the same meaning as in the earlier formula. Additionally, chemical transformations involving two or more reactants generally employ substantially stoichiometric amounts of each reactant, although certain reactions may employ an excess of one or more reactants to improve yield, etc.

As shown in Scheme I, the method also includes installing a protecting group, G, on the anilino nitrogen of Formula 5 to yield a protected 4-anilino-6-nitroquinazoline (Formula 6). Depending on the nature of the protecting group, G is installed using standard techniques such as acylation or alkylation. Generally, G may be any group used to protect an amine, including substituted or unsubstituted alkyl, alkenyl or benzyl. Other useful G include C(O)R¹⁰, COR¹⁰, CO₂R¹⁰, C(O)SₙR¹⁰, S(O)ₙR¹⁰, NHR¹⁰, NR¹⁰R¹¹, NHC(O)R¹⁰, OC(O)NHR¹⁰, OC(O)NHC(O)R¹⁰, OC(O)NR¹⁰R¹¹, C(O)R¹⁰Y, COR¹⁰Y, CO₂R¹⁰Y, C(O)SₙR¹⁰Y, S(O)ₙR¹⁰Y, NHR¹⁰Y, NHC(O)R¹⁰Y, OC(O)NHR¹⁰Y, or OC(O)NHC(O)R¹⁰Y, wherein Y is Si(R¹¹)₃, S(O)ₙR¹¹, OR¹¹, CN, NO₂, halogen, or P(O)(OR¹¹)₂, and R¹⁰ and R¹¹ are each, independently, substituted or unsubstituted C₁₋₆ alkyl, C₂₋₆ alkenyl, aryl or arylalkyl, and n is an integer between 0 and 2, inclusive. See Table II below for a list of useful protecting groups. For a more complete, but nonexclusive list of amine protecting groups, see T. W. Greene and P. G. Wuts, Protecting Groups in Organic Chemistry (1999), and P. Kocienski, Protective Groups (2000), noted above.

As indicated in Scheme I, the method includes reacting the protected 4-anilino-6-nitro-quinazoline of Formula 6 with hydrogen in the presence of a catalyst to give a 4-anilino-6-amino-quinazoline (Formula 7). The catalytic hydrogenation is carried out in a solvent and in the presence of a suitable catalyst, and may include an optional additive to reduce or prevent dehalogenation of the 4-anilino moiety. The reaction is typically carried out at elevated temperature (e.g., from about 70° C. to about 90° C.) under about 3 bar to about 10 bar H₂. Under these conditions, the 6-nitro-quinazoline of Formula 6 is often consumed after about 10 h, and in some cases, after about 4 h. Useful solvents include aprotic polar solvents, such as THF, DME, EtOAc, dioxane, and 2-methyltetrahydrofuran, and useful optional additives include P(OPh₃)₃, MgO, and morpholine. Suitable catalysts include heterogeneous catalysts such as Ir/C, Pd/V/C, Pt/Al₂O₃, Pt/Cu/C, Pt/graphite, Rh/Al₂O₃, IrO₂, PtO₂, Ru/C, Raney Ni, Pt/C, Rh/C, Pd/Fe/C, Pd/Ru/C, Pt/Fe/C, and Pt/V/C. Alternatively, the protected 4-anilino-6-nitro-quinazoline may be converted to the desired amine (Formula 7) using a reducing agent such as Fe/HCl, Fe/NH₄Cl, Zn/HCl, Sn/HCl, In/EtOH/NH₄Cl, Sm/I₂, Al(Hg)/THF, Et₃SiH/RhCl(PPh₃)₃, AlH₃-AlCl₃, HCO₂H/Pd/C, NaSH, NaBH₄/NiCl₂, or HCO₂NH₄/Pd/C.

Acryloylating the 6-amino group using an appropriate acylating agent (Formula 8 or Formula 9) results in an N-[4-anilino-quinazolin-6-yl] acrylamide (Formula 10). Useful acylating agents include activated forms of Formula 9 (e.g., acid halides, mixed anhydrides, and certain esters) in which X³ is a leaving group, including halogen, OC(O)R⁸, substituted or unsubstituted aryloxy (e.g. phenoxy), and heteroaryloxy (e.g., imidazolyloxy). Other suitable acylating agents include carboxylic acids of Formula 8, which are activated using a coupling agent.

Typically, the coupling reaction is carried out in an aprotic solvent, such as NMP, DMF, methylene chloride, etc., and may also employ a catalyst. Useful coupling agents include, but are not limited to DCC, FDPP, TATU, BOP, PyBOP, 1-(3-dimethylaminopropyl)-3-ethylcarbodiimide, diisopropyl carbodiimide, isopropenyl chloroformate, isobutyl chloroformate, N,N-bis-(2-oxo-3-oxazolidinyl)-phosphinic chloride, diphenylphosphoryl azide, diphenylphosphinic chloride, and diphenylphosphoryl cyanide. Useful catalysts for the coupling reaction include DMAP, HODhbt, HOBt, and HOAt.

In addition to α,β-unsaturated carbonyl compounds, suitable acylating agents may include saturated analogs (e.g., propionic acids or acid halides) of Formula 8 and Formula 9. In such cases, one would contact the resulting acylation product (not shown) with a strongly basic, hindered nucleophile, such as DABCO, DBU, DBN, t-BuOK, etc., to yield the desired acrylamide intermediate (Formula 10).

As shown in Scheme I, deprotecting the N-[4-anilino-quinazolin-6-yl] acrylamide of Formula 10 yields compounds of Formula 1. The reagents and conditions used to deprotect the compounds of Formula 10 will depend on the nature of the protecting group. Table II provides exemplary reagents and conditions for removing various protecting groups.

TABLE II

| Protecting Group, G | Reagents or Conditions |
|---|---|
| Ac | HCl |
| | TFA |
| allyl and allyloxycarbonyl | Rh(PPh₃)₃Cl |
| | Pd(PPh₃)₄, Bu₃SnH |
| | Pd(PPh₃)₄, dimedone |
| | Pd(PPh₃)₄, HCO₂H |
| Bn and Cbz | H₂, 10% Pd/C |
| | HCO₂H, Pd/C |
| | Raney Ni |
| Boc | TFA |
| | HCl |
| | Me₃SiI |
| | AlCl₃ |
| | Me₃SiOTf |
| | H₂SO₄ |
| 1,1,-dimethyl-2-cyanoethoxycarbonyl | aq K₂CO₃ |
| | Et₃N |
| 3,4-dimethoxybenzyl | TFA |
| | HCl |
| | methanesulfonic acid |
| 9-fluorenylmethylcarbamate | piperidine/DMF |
| | NH(i-Pr)₂/DMF |
| isopropyldithio carbonyl | mercaptoethanol |
| | NaOH |
| | Ph₃P, p-toluene sulfonic acid |
| MEM | ZnCl₂ |
| 4-methoxyphenacyl carbamate | photolysis |
| 2-methylsulfonylethoxycarbonyl | NaOH aq |
| m-nitrophenylcarbonyl | photolysis |
| 3-(o-nitrophenyl)propanamide | photolysis |
| 4-pentenyloxymethyl | I₂ |
| 9-phenylfluorenylmethylamine | TFA |
| 2-(2′-pyridyl)ethoxy carbonyl | t-BuOK, 18-crown-6 |
| 2,2,2-trichloroethylcarbamate | Zn, THF |
| | electrolysis |
| | Cd, AcOH |
| 2-trimethylsilylethyoxycarbonyl | Bu₄NF |
| | TFA |
| | ZnCl₂ |
| 2-trimethylsilylethoxymethyl | HCl |
| triphenylmethyl amine | HCl |
| | H₂, Pd black |
| | Na, NH₃ |

Though not shown in Scheme I, the method may include contacting the free base of Formula 1 with an acid to form an acid addition salt as described above. Since many of the deprotecting methods use an acid to cleave the protecting group from the anilino nitrogen, in some cases the formation of the acid addition salt may be combined with deprotection. Thus, for example, when G is an acetyl group, a compound of Formula 10 may be contacted with hydrochloric acid to remove G and to form a corresponding HCl salt.

Scheme II illustrates an alternative method for preparing the protected 4-anilino-6-nitroquinazoline of Formula 6. Instead of adding a protecting group following the formation of the 4-anilino-6-nitro-quinazoline of Formula 4, Scheme II reacts a substituted aniline (Formula 11) having a protected amine with the 4-substituted quinazoline of Formula 2 to yield an intermediate (Formula 12) that is subsequently reacted with an alcohol (R⁷OH), a thiol (R⁷SH), or a primary amine (R⁷NH₂) to yield the protected 4-anilino-6-nitroquinazoline of Formula 6. The protected 4-anilino-6-nitroquinazoline of Formula 6 then undergoes reaction in accordance with Scheme I to yield a desired compound of Formula 1 or its pharmaceutically acceptable salt.

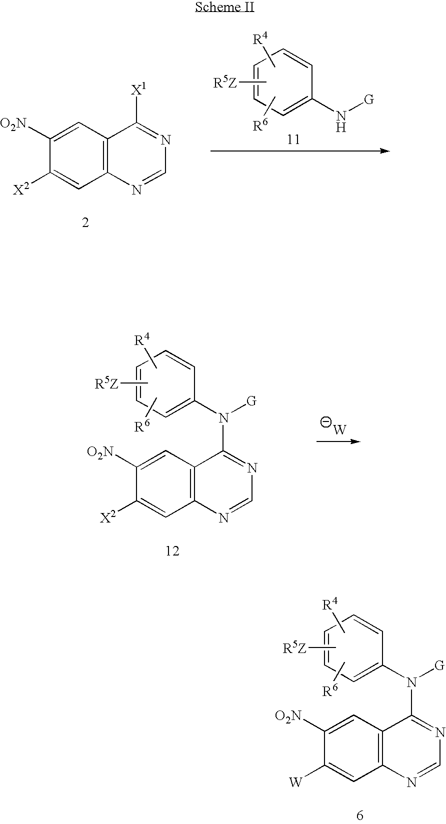

The protected aniline of Formula 11 may be prepared by alkylating or acylating a primary amine. For example, a phenylamine may be reacted with a carbonate derivative, such as Boc anhydride, Boc-ON, CbzCl, and R¹⁰C(O)Cl, to yield a corresponding N-phenyl-carbamate, where R¹⁰ is defined as above in Formula 6. Similarly, a phenylamine may be reacted with TFAA or a sulfonyl derivative, such as R¹⁰SO₂Cl, to yield an N-phenyl-trifluoroacetamide and an N-phenyl-sulfonamide, respectively. Particularly useful R¹⁰C(O)Cl and R¹⁰SO₂Cl include those in which R¹⁰ is t-butyl, allyl, benzyl, p-methoxybenzyl, 2-chloroethyl, 2,2,2-trichloroethyl, 2-trimethylsilyethyl, 2-nitroethyl, 2-cyanoethyl, 4-nitrobenzyl, trifluoromethyl, and the like.

Additionally, the protected aniline of Formula 11 may be obtained by reductive amination of a primary amine or aniline with an aldehyde (including but not limited to substituted and unsubstituted benzaldehydes) using a reducing reagent such as sodium cyanoborohydride or sodium triacetoxyborohydride. Thus, for example, 3-chloro-4-fluoro-aniline may be reacted with 3,4-dimethoxybenzaldehyde in the presence of NaBH(OC(O)CH₃)₃ to yield a protected aniline, (3-chloro-4-fluoro-phenyl)-(3,4-dimethoxy-benzyl)-amine.

A number of techniques can be used to attach the protected aniline to the 4-substituted quinazoline in Scheme II. For example, the protected aniline of Formula 11 may be coupled to the quinazoline of Formula 2 in the presence of a base using an optional transition metal catalyst. Useful couplings may employ 4-halogeno (e.g., 4-bromo) or 4-sulfonyloxy (e.g., 4-OTf) quinazolines and a catalyst comprised of a metal, such as Pd, Rh, or Cu, and a hindered phosphine ligand. The use of the latter quinazoline substrates and catalysts are often referred to as Buchwald couplings, and represent a favorable way to carry out this reaction.

Scheme III provides a useful method for preparing the quinazoline starting material (Formula 2) of Scheme I or Scheme II. The method includes reacting a substituted anthranilic acid (Formula 13) with excess formamidine acetate (e.g., two equivalents) to yield a quinazolin-4-one (Formula 14). The reaction is carried out at elevated temperature (e.g., 120° C.) in a protic solvent such as TEGMME. Other useful solvents include 2-methoxyethanol, NMP, and PGMME.

Following ring closure, the quinazolin-4-one of Formula 14 is nitrated using 65% nitric acid, yielding a mixture of 6-nitro (Formula 15) and 8-nitro-quinazolin-4-one isomers. One may obtain the desired 6-nitro isomer by crystallizing it from a suitable solvent, including DMF, HOAc, or NMP/EtOH. The reaction can be carried out at ambient temperature, but elevated temperatures (e.g., 60-70° C.) decrease reaction times from about 70 hours to about 6 hours without substantially affecting yields. A mixture of fuming nitric acid and concentrated sulfuric acid can also be used to nitrate the quinazolin-4-one, but the resulting isomeric mixture contains a comparatively large fraction of 8-nitro quinazolin-4-one (about a 25 wt. % as opposed to about 8-12 wt. % when using 65% HNO₃).

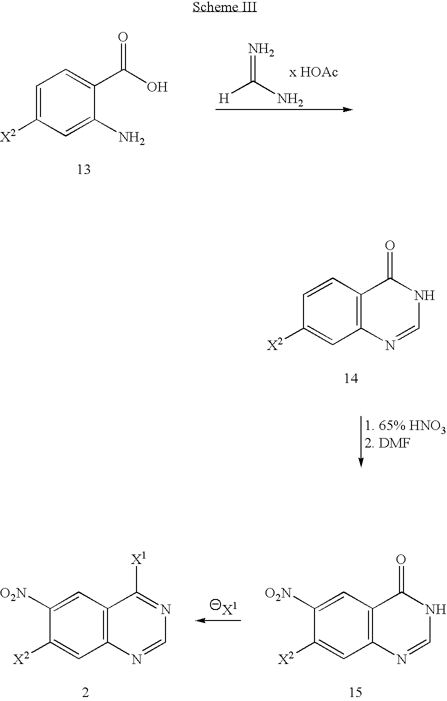

Following nitration, the 4-oxo moiety of Formula 15 is replaced with X¹ to give the activated quinazoline starting material of Formula 2. As indicated above, particularly useful compounds of Formula 2 include 4-chloro quinazolines, which may be prepared by direct conversion of the quinazilone of Formula 14 using POCl₃ or SOCl₂.

Instead of formamidine acetate, one may use formamide or s-triazine in the ring closure reaction of Scheme III. Both reagents provide certain advantages over formamidine acetate. For example, formamide is a liquid and therefore easier to handle than formamidine acetate, and reactions using s-triazine can be carried out in ethanol instead of TEGMME and the like. However, conversions using formamide may require a substantial excess of formamide (e.g., five equivalents) to effect yields comparable to formamidine acetate. S-triazine is more costly than formamidine acetate, but one may obtain good yields using stoichiometric amounts.

As indicated above, Scheme I and Scheme II reduce the risk of acryloylation of the 4-anilino nitrogen through the use of a protecting group, G, which is subsequently removed to yield compounds of Formula 1. Another way to avoid the formation of unwanted diacryloylamino side products is to install the 6-acryloyl side chain on the quinazoline nucleus before attachment of the 4-anilino group. One potential problem with this strategy, however, is degradation of the 6-acryloyl group under conditions needed for introduction of the anilino group.

Scheme IV provides a method for preventing diacryloylamino side products by installing the aniline (Formula 3) after the attachment of the acrylamide group. The acrylamide substituent is masked (protected) so that it remains intact under conditions employed to install the anilino group. The method uses some of the same steps depicted in Scheme I and Scheme II, and thus includes reacting a 6-nitro-quinazoline-4-one (Formula 15) with an alcohol (R⁷OH), a thiol (R⁷SH), or a primary amine (R⁷NH₂) in the presence of a strong base to yield a 6-nitro-quinazolin-4-one (Formula 16) having a 7-oxy, sulfanyl or amino-side chain (W). As in Scheme III, the 4-oxo moiety of the 7-substituted-6-nitro-quinazilone of Formula 16 is replaced with X¹ to give an activated quinazoline of Formula 17, which is reacted with hydrogen in the presence of a catalyst to give a 7-substituted-6-amino-quinazoline (Formula 18). Alternatively, and as noted above in discussing Scheme I, the 7-substituted-6-nitro-quinazoline may be converted to the compound of Formula 18 using a suitable reducing agent.

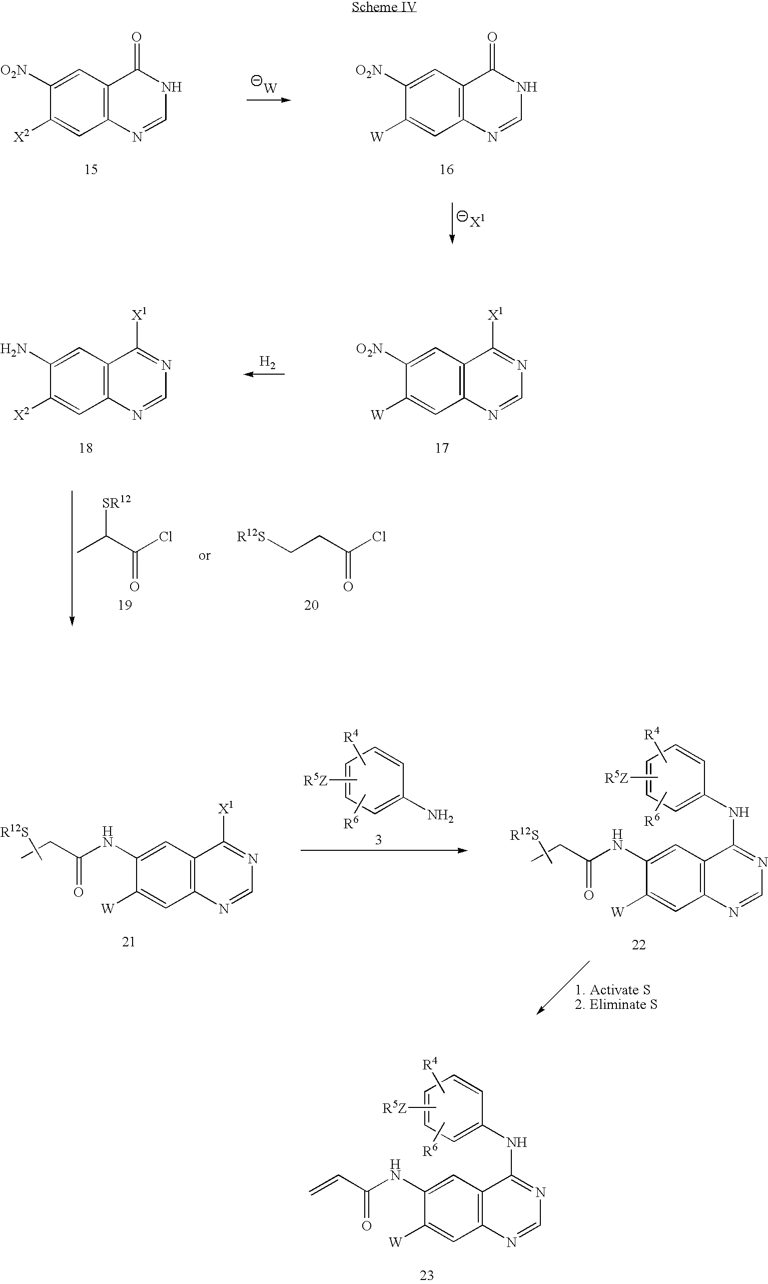

The method shown in Scheme IV includes acylating the 6-amino substituent of the compound of Formula 18 using a 2- or 3-sulfanyl-proprionyl chloride (Formula 19 or 20) to yield 2- or 3-sulfanyl-N-quinazolin-6-yl-propionamide (Formula 21). Following preparation of the masked acrylamides of Formula 21, the anilino group (Formula 3) is installed using methods described elsewhere in this disclosure to yield a 4-anilino-quinazoline (Formula 22). To reveal the acrylamide of Formula 23, the sulfur atom of the 4-anilino-quinazoline of Formula 22 is activated by, for example, oxidizing the 2-sulfanyl-propionamide to a sulfoxide or oxidizing the 3-sulfanyl to a sulfoxide or a sulfone. The resulting 2-sulfinyl-propionamide and 3-sulfinyl or 3-sulfonyl-propionamide undergo facile thermal elimination or mild base elimination, respectively, to give the unmasked acrylamide of Formula 23. In Formula 19 and Formula 20, useful R¹² include, but are not limited to C₁₋₆ alkyl (e.g., Me, i-Pr, t-Bu) and aryl (e.g., Ph).

Scheme V shows another method for masking the 6-acryloyl side chain using a diacyloxydiazaoxabicycloheptane (DADAOB). The method includes attaching the DADAOB-protected form of the 6-acryloyl side chain (Formula 24 or Formula 25) to a 7-substituted-6-amino-quinazoline (Formula 18), which results in a quinazolin-6-yl-amide (Formula 26). Useful DADAOB-protected forms include activated moieties of Formula 25 (e.g., acid halides, mixed anhydrides, and certain esters) in which X⁴ is a leaving group, including halogen, OC(O)R⁸, substituted or unsubstituted aryloxy (e.g. phenoxy), and heteroaryloxy. Other suitable DADAOB-protected forms include carboxylic acids of Formula 24, which are activated using a coupling agent.

Typically, the coupling reaction shown in Scheme V is carried out in an aprotic solvent, such as NMP, DMF, methylene chloride, etc., and may also employ a catalyst. Useful coupling agents and catalysts include those described in connection with the coupling of the 6-acryloyl group to compounds of Formula 7 (Scheme I). Suitable R¹³ in Formula 24 and Formula 25 include C₁₋₄ alkyl (e.g., Me, Et, n-Pr, i-Pr), C₁₋₄ haloalkyl (e.g., chloroethyl, 2,2,2-trichloroethyl, bromoethyl), C₂₋₄ alkenyl (e.g., allyl), TMS-(CH₂)ₘ or aryl-(CH₂)ₘ (e.g., Bn). Additionally, R¹⁴ may include hydrogen, halogen, C₂₋₆ alkenyl, C₂₋₆ alkynyl, and C₂₋₆ alkenyl or C₂₋₆ alkynyl substituted with hydroxy, alkoxy, amino or alkylamino.

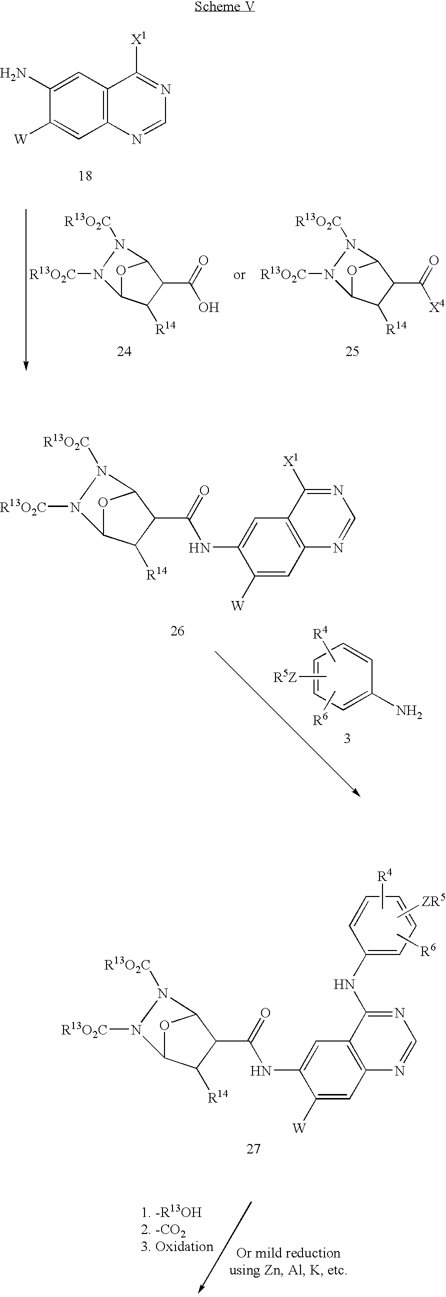

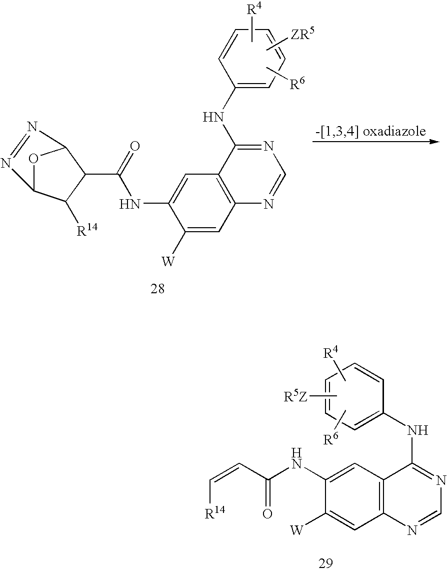

Following preparation of the masked acrylamides of Formula 26, the anilino group (Formula 3) is installed using methods disclosed above to yield a 4-anilino-quinazoline (Formula 27). To unmask the acrylamide, R¹³ can be cleaved through acid- or base-catalyzed hydrolysis of the DADAOB ester moieties (CO₂R¹³) to yield R¹³OH and a quinazolin-6-yl dicarboxylic acid. The latter intermediate can be decarboxylated by, for example, heating in the presence of an acid, to give a 6-(7-oxa-2,3-diaza-bicyclo[2.2.1]heptane-5-carbonylamino)-quinazoline, which is subsequently oxidized using a mild oxidizing agent (e.g., t-BuOCl, NaOBr, HgO, K₃Fe(CN)₆, MnO₂, CuCl₂, air and NaOH) to yield an azo-compound of Formula 28. Alternatively, in certain cases (e.g., when R¹³ is a chloro-substituted ethyl) the DADAOB moiety in Formula 27 may be converted directly to the diazaoxabicycloheptene group in Formula 28 using mild reducing agents (Zn, Al, K). In either case, a retro-Diels-Alder reaction generates an unmasked acrylamide (Formula 29) as well as [1,3,4]oxadiazole.

Scheme VI provides a method for preparing DADAOB-CO₂H (Formula 24) and DADAOB-C(O)X⁴ (Formula 25). The method includes reacting an azocarboxylate (Formula 30) with a furan-3-yl-carboxylic acid or carboxylic acid methyl ester (i.e., R¹⁵ is H or Me in Formula 31) to yield a DADAOB intermediate (Formula 32). Following the Diels-Alder thermal [4+2] cycloaddition, the DADAOB intermediate is reacted with hydrogen in the presence of a Pd catalyst to yield (upon treatment with LiOH if R¹⁵ is not H) DADAOB-CO₂H (Formula 24). The activated forms of DADAOB (Formula 25) may be prepared from DADAOB-CO₂H using standard techniques (e.g., reaction with SOCl₂ or BBr₃/Al₂O₃).

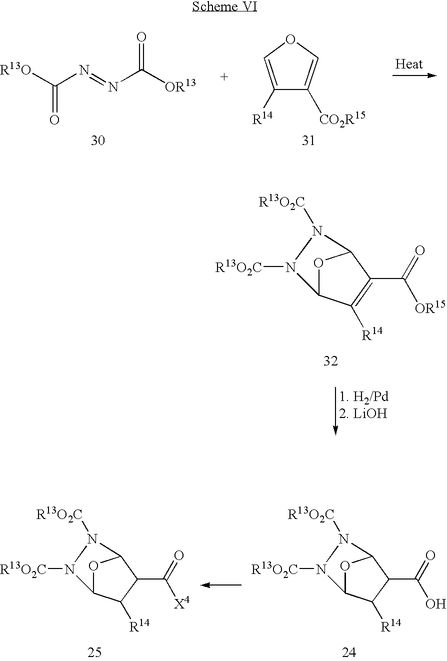

Scheme VII and Scheme VIII illustrate other methods for preparing masked acrylamides using the DADAOB-protected forms (Formula 24 or Formula 25). Like Scheme IV, Scheme VII reacts a 6-nitro-quinazoline-4-one (Formula 15) with an alcohol (R⁷OH), a thiol (R⁷SH), or a primary amine (R⁷NH₂) in the presence of a strong base to yield a 6-nitro-quinazolin-4-one (Formula 16) having a 7-oxy, sulfanyl or amino-side chain (W). The resulting 7-substituted-6-nitro-quinazilone of Formula 16 is subsequently reacted with hydrogen in the presence of a catalyst (e.g., Pd/C) to give a 7-substituted-6-amino-quinazoline (Formula 33). Following reduction of the 6-nitro group, the 7-substituted-6-amino-quinazoline of Formula 33 is reacted with the DADAOB-protected forms of the 6-acryloyl side chain (Formula 24 or Formula 25) to yield a quinazolin-6-yl-amide of Formula 34.

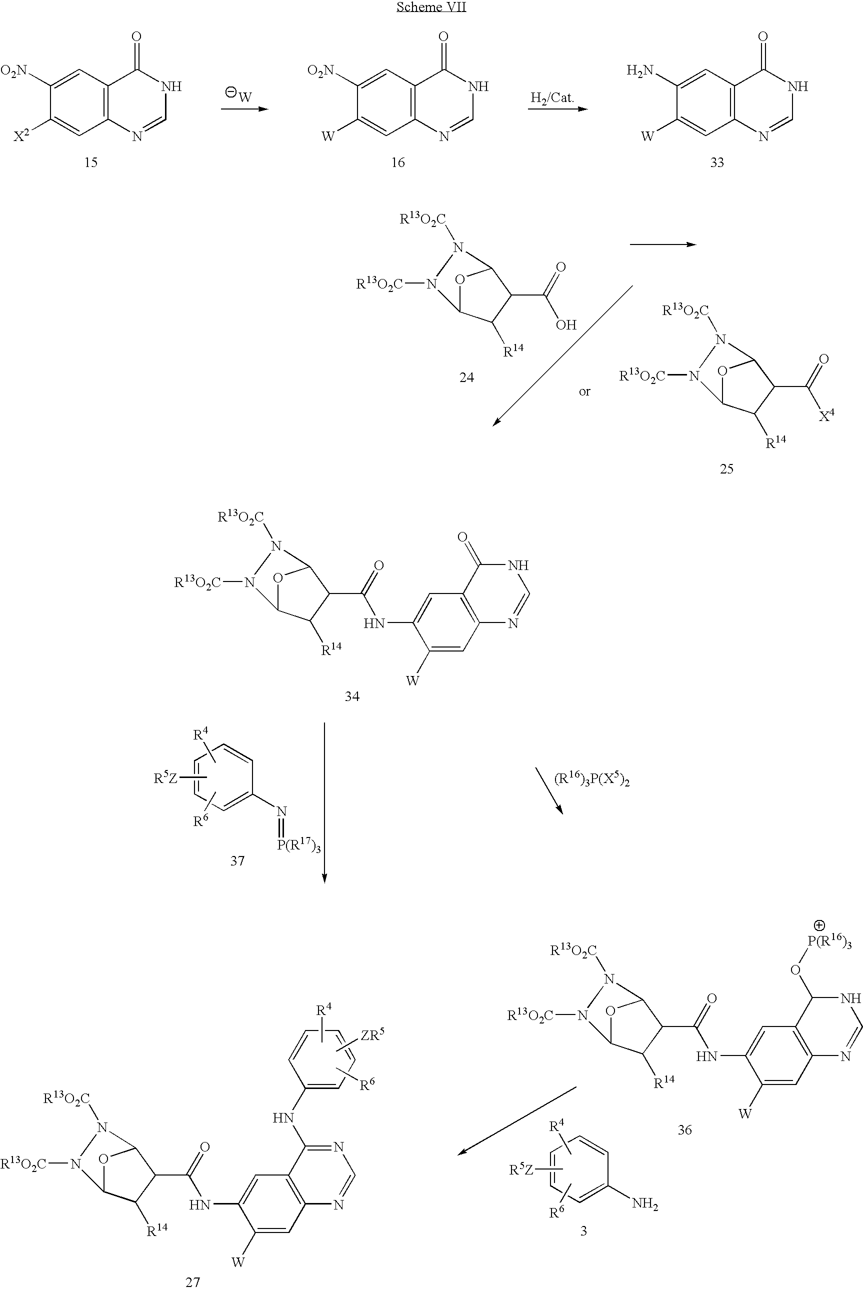

Though other techniques may be used, the method shown in Scheme VII employs a phosphine-induced coupling to convert the quinazolin 6-yl-amide of Formula 34 to the unmasked acrylamide of Formula 29. The compound of Formula 34 is reacted with a phosphorus-containing dehydrating agent (Formula 35) to yield a 4-oxyphosphonium quinazoline (Formula 36), which is subsequently reacted with an aniline (Formula 3) to yield a 4-anilino-quinazoline (Formula 27). Alternatively, the amine (Formula 3) may be converted to an iminophosphorane (Formula 37), which is subsequently reacted with the quinazolin 6-yl-amide of Formula 34 to yield the 4-anilino-quinazoline of Formula 27 directly. The iminophosphorane of Formula 37 may be prepared by methods that include, for example, conversion to a corresponding azide, followed by reaction with an appropriate phosphine. Following the phosphine-induced coupling, the 4-anilino-quinazoline of Formula 27 is converted to the unmasked acrylamide of Formula 29 in the manner shown in Scheme V.

In Scheme VII, useful phosphorus-containing dehydrating agents include, without limitation, triphenylphosphine dihalides, triphenylphosphite dihalides, tributylphosphine dibromide, Ph₃P with a dialkylazodicarboxylate such as DEAD (Mitsunobu conditions), and bis(triphenylphosphine)oxide triflate. In Formula 35 and Formula 37, R¹⁶ and R¹⁷ may be, but are not limited to C₁₋₆ alkyl, phenyl or phenoxy, and X⁵ is hydrogen, halogen or absent.

Scheme VIII shows a method for preparing masked acrylamides, which utilizes a Buchwald coupling to install the 4-anilino group. The method includes reacting a 4-aminoquinazoline having a DADAOB-protected acryloyl side chain (Formula 38) with an aryl halide or O-arylsulfonate (Formula 39) to yield a 4-anilino-quinazoline of Formula 40. The reaction is carried out in the presence of a catalyst, which is comprised of a transition metal (e.g., Pd, Rh or Cu) and a hindered phosphine ligand (e.g., bis-di-tert-butyl-1-biphenylphosphine). Following the Buchwald coupling, the 4-anilino-quinazoline of Formula 40 is converted to the unmasked acrylamide of Formula 29 in the manner depicted in Scheme V. The 4-aminoquinazoline of Formula 38 may be prepared using methods known in the art or through chemical transformations analogous to those shown in Scheme IV, in which one substitutes the masked acrylamides of Formula 19 and 20 with those of Formula 24 and 25, and replaces the amine of Formula 3 with an amine having the formula R¹⁸NH₂.

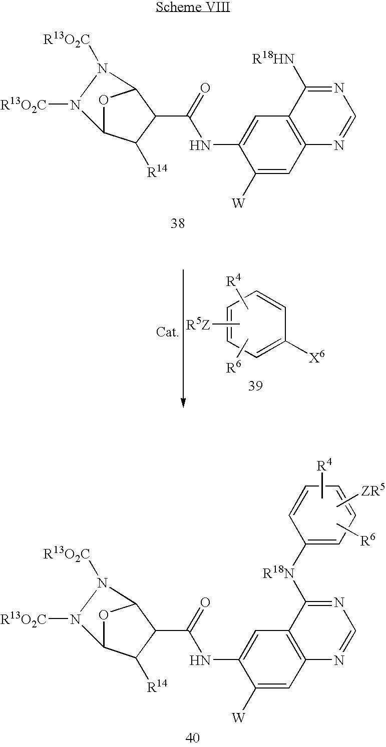

As indicated above, substituent X⁶ in Formula 39 is halogen (especially Br or I) or O-sulfonate (e.g., TfO). In Formula 38, substituent R¹⁸ can be hydrogen, but may also be a group that facilitates the coupling of the aryl halide or O-arylsulfonate to the 4-aminoquinazoline substrate. Such groups would be removed following the coupling reaction, and include, but are not limited to, O-substituted carbonyldioxy radicals or S-substituted sulfonyl radicals having t-butyl, allyl, benzyl, p-methoxybenzyl, 2-chloroethyl, 2,2,2-trichloroethyl, 2-trimethylsilylethyl, 2-nitroethyl, 2-cyanoethyl, 4-nitrobenzyl, trifluoroacetyl or Tf substituents. Following the coupling reaction, the 4-anilino-quinazoline of Formula 40 may be treated with an acid (e.g., dilute HCl) to remove non-hydrogen R¹⁸.

Many of the methods that employ DADAOB-masked acrylamides (Scheme V-Scheme VIII) may use other acryloyl-masked side chains, and depending on the conditions used to couple the amine (Formula 3, 37 and 38), may potentially use unprotected 6-acryloyl groups. One may also modify and combine the disclosed schemes so that they employ masked acryloyl side chains and protected anilino groups.

Scheme IX shows another method for minimizing undesirable diacryloylation, which may be used in place of, or in addition to, the protection schemes described elsewhere in this disclosure. The method comprises installing bulky groups at one or both ring positions that are adjacent (ortho) to the amino substituent of the protected aniline of Formula 11 (see Scheme II) prior to attaching the aniline to the quinazoline of Formula 3. Thus, for example, the method may include brominating the 6-position of the protected aniline of Formula 11, in which R⁴, ZR⁵ and R⁶ are chlorine, fluorine and hydrogen (Formula 41), to yield a 6-bromo-2-chloro-3 fluoro-aniline of Formula 42. A bulky silyl group is installed by first reacting the 6-bromo-aniline of Formula 42 with s-BuLi to effect a bromine-lithium exchange, and subsequently reacting the phenyl lithium intermediate with (R¹⁹)₃SiCl to yield a silylamine of Formula 43. Suitable R¹⁹ include, without limitation, C₁₋₄ alkyl (e.g., Me, Et, i-Pr, t-Bu), C₁₋₄ alkoxy, and aryl (e.g., phenyl, substituted phenyl).

Following deprotection of the amino moiety, and using methods described above, the silylamine is coupled to the quinazoline starting material of Formula 2, and the resulting 4-(6-silyl-anilino)-quinazoline (Formula 44) undergoes further reaction to yield a quinazoline (Formula 45) having a 6-acryloylamino and 7-oxy-, sulfanyl- or amino-side chains (W). When the bulky silyl group is no longer needed, it can be displaced with hydrogen by treatment with an acid to yield a desired 6-acryloylamino-4-anilino-7-(oxy, sulfanyl or amino)-quinazoline (Formula 46). Other embodiments may utilize two silyl groups by brominating the 2- and 6-positions of the protected aniline of Formula 11.

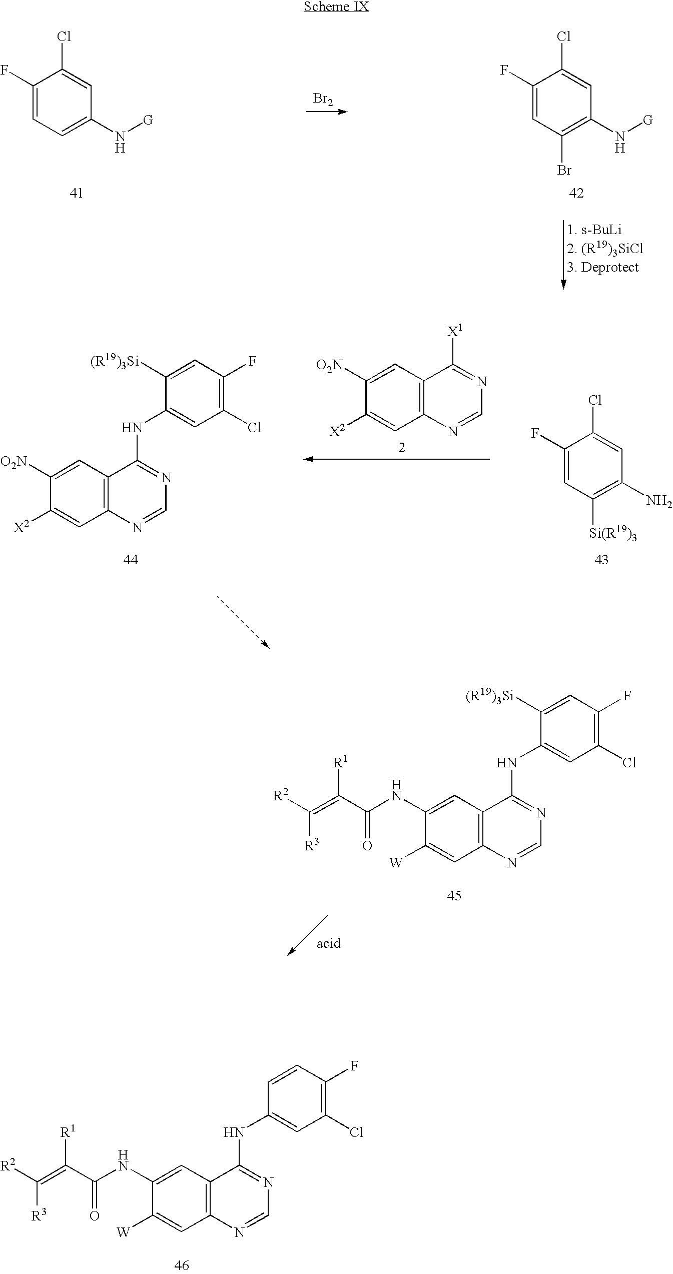

Many of the compounds described in this disclosure, including those represented by Formula 1, are capable of forming pharmaceutically acceptable salts. These salts include, without limitation, acid addition salts (including diacids) and base salts. Pharmaceutically acceptable acid addition salts include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like.

Pharmaceutically acceptable base salts include nontoxic salts derived from bases, including metal cations, such as an alkali or alkaline earth metal cation, as well as amines. Examples of suitable metal cations include, without limitation, sodium cations (Na⁺), potassium cations (K⁺), magnesium cations (Mg²⁺), calcium cations (Ca²⁺), and the like. Examples of suitable amines include, without limitation, N,N′-dibenzylethylenediamine, chloroprocaine, choline, diethanolamine, dicyclohexylamine, ethylenediamine, N-methylglucamine, and procaine. For a discussion of useful acid addition and base salts, see S. M. Berge et al., “Pharmaceutical Salts,” 66 J. of Pharm. Sci., 1-19 (1977); see also Stahl and Wermuth, Handbook of Pharmaceutical Salts: Properties, Selection, and Use (2002).

One may prepare a pharmaceutically acceptable acid addition salt (or base salt) by contacting a compound's free base (or free acid) with a sufficient amount of a desired acid (or base) to produce a nontoxic salt. One may then isolate the salt by filtration if it precipitates from solution, or by evaporation to recover the salt. One may also regenerate the free base (or free acid) by contacting the acid addition salt with a base (or the base salt with an acid). Though certain physical properties of the free base (or free acid) and its respective acid addition salt (or base salt) may differ (e.g., solubility, crystal structure, hygroscopicity, etc.), a compound's free base and acid addition salt (or its free acid and base salt) are otherwise equivalent for purposes of this disclosure.

Additionally, certain compounds of this disclosure, including those represented by Formula 1, may exist as an unsolvated form or as a solvated form, including hydrated forms. Pharmaceutically acceptable solvates include hydrates and solvates in which the crystallization solvent may be isotopically substituted, e.g. D₂O, d₆-acetone, d₆-DMSO, etc. Generally, the solvated forms, including hydrated forms, are equivalent to unsolvated forms for the purposes of this disclosure. Thus, unless expressly noted, all references to the free base, the free acid or the unsolvated form of a compound also includes the corresponding acid addition salt, base salt or solvated form of the compound.

Some of the compounds disclosed in this specification may also contain one or more asymmetric carbon atoms and therefore may exist as optically active stereoisomers (i.e., pairs of enantiomers). Some of the compounds may also contain an alkenyl or cyclic group, so that cis/trans (or Z/E) stereoisomers (i.e., pairs of diastereoisomers) are possible. Still other compounds may exist as one or more pairs of diastereoisomers in which each diastereoisomer exists as one or more pairs of enantiomers. Finally, some of the compounds may contain a keto or oxime group, so that tautomerism may occur. In such cases, the scope of the present invention includes individual stereoisomers of the disclosed compound, as well as its tautomeric forms (if appropriate).

Individual enantiomers may be prepared or isolated by known techniques, such as conversion of an appropriate optically-pure precursor, resolution of the racemate (or the racemate of a salt or derivative) using, for example, chiral HPLC, or fractional crystallization of diastereoisomeric salts formed by reaction of the racemate with a suitable optically active acid or base (e.g., tartaric acid). Diastereoisomers may be separated by known techniques, such as fractional crystallization and chromatography.

The disclosed compounds also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as ³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### EXAMPLES

The following examples are intended to be illustrative and non-limiting, and represent specific embodiments of the present invention.

### Example 1

### Preparation of (3-chloro-4-fluoro-phenyl)-(3,4-dimethoxy-benzyl)-amine

Procedure A. 3-Chloro-4-fluoro-aniline (5.00 g, 34.35 mmol) and 3,4-dimethoxy benzaldehyde (6.28 g, 37.78 mmol) were combined in glacial acetic acid (17 mL) warming slightly to dissolve all the solids. To the resulting orange colored solution was added with stirring 4A Mol sieves (5.00 g) and the mixture was stirred overnight. Additional glacial acetic acid (25 mL) was added, followed by sodium triacetoxy borohydride (8.74 g, 41.22 mmol). A thick precipitate formed after stirring for about 1 h. The solids were collected by filtration, and the filtrate was set aside. The solids were treated with 1 N sodium hydroxide (aq). The resulting solution was extracted with ethyl acetate. The insoluble solids (sieves) were washed with hot ethyl acetate. The combined organic layers were dried over magnesium sulfate, filtered and evaporated to give (3-chloro-4-fluoro-phenyl)-(3,4-dimethoxy-benzyl)-amine (5.69 g) as a yellow orange solid. The filtrate previously set aside was evaporated in vacuo and the residue was treated with 1 N sodium hydroxide (aq) and extracted with ethyl acetate. This extract was dried over magnesium sulfate, filtered, and evaporated in vacuo to give a sticky orange brown solid that contained some un-reacted 3,4-dimethoxy benzaldehyde. Chromatography on a BIOTAGE 40M cartridge eluted with 1:1 hexanes:methylene chloride gave additional (3-chloro-4-fluoro-phenyl)-(3,4-dimethoxy-benzyl)-amine (3.37 g) for a total of 9.06 g (89%). Melting point 127-128° C.; ¹H NMR (400 MHz, CDCl₃): δ 6.93 (t, 1H, J=8.91 Hz), 6.81-6.88 (m, 3H,), 6.64 (q, 1H, J=3.17 Hz), 6.45 (dd, 1H, J=3.17, 0.49 Hz ArH), 4.18 (s 2H, CH₂), 3.86 (d, 6H, J=1.47 Hz, CH₃O), 1.55 (bs, 1H, NH); ¹⁹F NMR (376.3 MHz, CDCl₃): −131.20 (bs).

Procedure B. A mixture of 3-chloro-4-fluoroaniline (75.0 g, 515 mmol), 3,4-dimethoxybenzaldehyde (85.6 g, 515 mmol) and isopropyl alcohol (755 mL) was stirred until a homogeneous solution was obtained. After cooling to −1° C. ±2° C., acetic acid (31.1 g, 518 mmol) was added to the reaction mixture followed by sodium cyanoborohydride (38.9 g, 619 mmol). The reaction mixture was stirred at ambient temperature until completion of the reaction (3-4 h). The reaction was quenched with 1 N NaOH (aq) (515 mL) and the resulting slurry cooled to 0° C., held for 20-30 min, then filtered and washed with water until the pH of the product cake was neutral. The product cake was dried in a vacuum oven at 50° C. to yield (3-chloro-4-fluoro-phenyl)-(3,4-dimethoxy-benzyl)-amine (143.3 g, 94%).

### Example 2

### Preparation of 4-chloro-7-fluoro-6-nitro quinazoline

7-Fluoro-6-nitro-3H-quinazolin-4-one (30.00 g, 143.45 mmol) was suspended at room temperature in thionyl chloride (80 mL) with stirring, followed by addition of dimethyl formamide (1.5 mL). The resulting thick suspension was heated to reflux for 18 h by which time the suspension had dissolved resulting in a yellow solution. Distillation of the excess thionyl chloride at reduced pressure resulted in a brown syrup that was azeotroped twice with toluene to remove excess thionyl chloride. The resulting brown solids were then dissolved in a minimum amount of methylene chloride and rapidly filtered through a thin pad of silica, washing the pad with additional methylene chloride (˜0.5 L). The resulting yellow filtrate was evaporated under reduced pressure to give 4-chloro-7-fluoro-6-nitro quinazoline (32.45 g, 99%). Melting point 116-118° C.; ¹H NMR (400 MHz, CDCl₃): δ 9.12 (s, 1H), 8.98 (d, 1H, J=7.57 Hz), 7.89 (d, 1H, J=10.75 Hz); ¹⁹F NMR (376.3 MHz, CDCl₃): δ −109.02 (t, J=10.11 Hz); MS (APCI−) m/z 227 (M−1).

### Example 3

### Preparation of (3-chloro-4-fluoro-phenyl)-(3,4-dimethoxy-benzyl)-(7-fluoro-6-nitro-quinazolin4-yl)-amine

Procedure A. To a suspension of (3-chloro-4-fluoro-phenyl)-(3,4-dimethoxy-benzyl)-amine (3.86 g, 1.50 mmol) in isopropanol (52 mL) was added 4 chloro-7-fluoro-6-nitro-quinazoline (1.41 g, 0.72 mmol). The resulting suspension was heated to reflux for 1 h, then the heat was removed and the reaction was allowed to stand and cool overnight at −10° C. The resulting thick precipitate was filtered, and the solids were washed with additional isopropanol and allowed to dry in the filter funnel. The yellow filtrate was concentrated under reduced pressure to give a solid. The combined solids were dissolved in a minimum amount of methylene chloride and placed on a 90 cm diameter by 40 cm thick pad of silica eluting with approximately 1 L of methylene chloride to remove the excess (3-chloro-4-fluoro-phenyl)-(3,4-dimethoxy-benzyl)-amine. The desired product was eluted from the silica with 2% methanol in methylene chloride, and the eluent was evaporated under reduced pressure to give a bright yellow glass. Treatment of this material with diethyl ether and ultrasonication gave a yellow solid that was filtered and washed with small amounts of diethyl ether. Upon drying in vacuo, this solid gave (3-chloro-4-fluoro-phenyl)-(3,4-dimethoxy-benzyl)-(7-fluoro-6-nitro-quinazolin-4-yl)-amine (2.42 g, 86%). ¹H NMR (400 MHz, DMSO-d₆): δ 7.80 (d, 2H, J=9.0 Hz), 7.72 (d, 1H, J=9.0 Hz), 7.48 (t, 1H, J=9.0 Hz), 7.32 (m, 1H), 6.93 (d, 1H, J=1.9 Hz), 6.79 (d, 2H, 1.9 Hz), 5.38 (s, 2H, CH₂), 3.65 (s, 3H, OCH₃), 3.63(s 3H, OCH₃); ¹⁹F NMR (376.3 MHz, CDCl₃): δ −108.82 (s), −102.51 (s); MS (APCI−) m/z 488, 486 (M−1).

Procedure B. (3-Chloro-4-fluoro-phenyl)-(3,4-dimethoxy-benzyl)-amine (27.4 g, 92.6 mmol) and 4-chloro-7-fluoro-6-nitro-quinazoline (21.1 g, 92.6 mmol) were slurried in acetonitrile (200 mL). The yellow suspension was heated to 75° C. for 3 h. The heat was removed and the reaction was allowed to cool to room temperature with stirring overnight. The thick slurry was further cooled to 5° C. and K₂CO₃ (15.8 g, 115 mmol) dissolved in water (250 mL) was charged to the reaction, keeping the temperature <5° C. during the addition. The yellow slurry was stirred at 3-5° C. for an additional 30 min. The yellow solid was filtered and the cake washed with water (2×80 mL). The cake was dried at 50° C. under vacuum for 24 h to yield (3-chloro-4-fluoro-phenyl)-(3,4-dimethoxy-benzyl)-(7-fluoro-6-nitro-quinazolin-4-yl)-amine with a 2% impurity of (3-chloro-4-fluoro-phenyl)-(3,4-dimethoxy-benzyl)-amine by HPLC area % (41.7 g, 88% corrected yield). The product was used in subsequent transformations without purification. Melting point 136-138° C.; ¹H NMR (400 MHz, DMSO), δ 9.02 (s, 1H), 7.92 (d, 1H), 7.84 (m, 1H), 7.72 (d, 1H), 7.56 (m, 1H), 7.42 (m, 1H), 6.96 (s, 1H), 6.83 (s, 2H), 5.48 (s, 2H, BnCH₂N), 3.70 (s, 3H, OCH₃), 3.68 (s, 3H, OCH₃).

### Example 4

### Preparation of (3-chloro-4-fluoro-phenyl)-(3,4-dimethoxy-benzyl)-[7-(3-morpholin-4-yl-propoxy)-6-nitro-quinazolin-4-yl]-amine

Procedure A. (3-Chloro-4-fluoro-phenyl)-(3,4-dimethoxy-benzyl)-(7-fluoro-6-nitro-quinazolin-4-yl)-amine (0.20 g, 0.41 mmol) and morpholin-4-yl-propan-1-ol (0.060 g, 0.41 mmol) were suspended together in THF/t-BuOH (2:1, 3 mL) and cooled to 5° C. in an ice-salt bath. Potassium t-butoxide (0.05 g, 0.41 mmol) was added as a solid with vigorous stirring, resulting in an orange-brown colored mixture. The ice bath was removed after 1 h and the reaction mixture was stirred for 12 to 18 h at room temperature. The THF/t-BuOH was removed under reduced pressure; ethyl acetate and saturated aqueous sodium bicarbonate were added and the mixture was shaken. The layers then were separated and the aqueous layer was re-extracted with ethyl acetate. The pooled ethyl acetate layers were washed once with brine, and dried over magnesium sulfate. Filtration and evaporation of the solvent under reduced pressure provided the crude product as a bright yellow glass. Chromatography on a BIOTAGE 12M cartridge eluted with 5% MeOH in methylene chloride gave (3-chloro-4-fluoro-phenyl)-(3,4-dimethoxy-benzyl)-[7-(3-morpholin-4-yl-propoxy)-6-nitro-quinazolin-4-yl]-amine (0.122 g, 49%). Melting point 129-132° C.; ¹H NMR (400 MHz, CDCl₃): δ 8.81 (s, 1H), 7.52 (s, 1H), 7.31 (s, 1H), 7.17 (m, 2H), 6.94 (m, 2H), 6.76 (m, 2H), 5.31 (s, 2H, BnCH₂N), 4.25 (t, 2H, J=6.1 Hz, 0CH₂), 3.84 (s, 3H, OCH₃), 3.79 (s, 3H, OCH₃), 3.74 (bs 4H, CH₂OCH₂), 2.54 (bd 6H, NCH₂), 2.08 (bs, 2H, CH₂CH₂CH₂); MS (APCI+) m/z 612.2 (M+1).

Procedure B. (3-Chloro-4-fluoro-phenyl)-(3,4-dimethoxy-benzyl)-(7-fluoro-6-nitro-quinazolin-4-yl)-amine (40.6 g, 83.3 mmol) was dissolved in acetonitrile (400 mL). To the mixture was added morpholin-4-yl-propan-1-ol (12.1 g, 83.3 mmol) and the resulting orange-yellow solution was cooled to −15° C. Sodium t-butoxide (9.6 g, 100 mmol) was charged slowly as a solid to the reaction mixture and the resulting dark red solution was stirred for 4 h while maintaining the temperature between −20° C. and −10° C. Water (1 L) was charged to the reaction slowly while keeping the temperature less than 5° C. The resulting yellow suspension was stirred for 1 h. The precipitate was filtered and washed with water (125 mL). After drying at room temperature under nitrogen flow overnight, the crude cake was heated to 65° C. in i-Pr alcohol (700 mL) to obtain a dark homogenous solution. The mixture was slowly cooled to 0° C. to initiate crystallization (at about 35° C.), was held at 0° C. for about 1 h, then filtered and washed with cold i-Pr alcohol (2×60 mL). The cake was dried at 50° C. under vacuum for 24 h to give (3-chloro-4-fluoro-phenyl)-(3,4-dimethoxy-benzyl)-[7-(3-morpholin-4-yl-propoxy)-6-nitro-quinazolin-4-yl]-amine as a yellow solid (34.0 g, 67%). Melting point 135-138° C.; ¹H NMR (400 MHz, DMSO): δ 8.80 (s, 1H), 7.72 (m, 1H), 7.45 (m, 3H), 7.28 (m, 1H), 7.00 (s, 1H), 6.83 (d, 2H), 5.37 (s, 2H, BnCH₂N), 4.30 (t, 2H, OCH₂), 3.69 (s, 3H, OCH₃), 3.66 (s, 3H, OCH₃), 3.55 (t, 4H, O(CH₂)₂), 2.50 (t, 2H, NCH₂), 2.43 (bs, 4H, N(CH₂)₂), 1.99 (bs, 2H, CH₂CH₂CH₂).

### Example 5

### Preparation of N4-(3-chloro-4-fluoro-phenyl)-N4-(3,4-dimethoxy-benzyl)-7-(3-morpholin-4-yl-propoxy)-quinazoline-4,6-diamine

Procedure A. (3-Chloro-4-fluoro-phenyl)-(3,4-dimethoxy-benzyl)-[7-(3-morpholin-4-yl-propoxy)-6-nitro-quinazolin-4-yl]-amine (0.35 g, 0.57 mmol) was dissolved in THF (16 mL) in a Parr shaker bottle. Raney Nickel (0.30 g) was added. The mixture was then subjected to hydrogen at 40 psig for 17.5 h. The reaction mixture was filtered through Celite to remove the catalyst and the resulting filtrate was evaporated under reduced pressure to give N4-(3-chloro-4-fluoro-phenyl)-N4-(3,4-dimethoxy-benzyl)-7-(3-morpholin-4-yl-propoxy)-quinazoline-4,6-diamine (0.32 g, 96%) as a white foam. ¹H NMR (400 MHz, CDCl₃): δ 8.70 (s, 1H), 7.18 (s, 1H), 7.07 (dd, 1H, J=2.68, 6.34 Hz), 6.97 (m, 2H), 6.83 (dd, 1H, J=1.71, 8.30 Hz), 6.76 (m, 2H), 6.30 (s, 1H), 4.21 (t, 2H J=6.10 Hz), 4.00 (bs, 2H), 3.83 (s, 3H, OCH₃), 3.77 (s, 3H, OCH₃), 3.74 (m, 4H, CH₂OCH₂), 2.59 (bt, 2H), 2.52 (bs, 2H, NCH₂), 2.10 (m, 2H, CH₂CH₂CH₂), 1.84 (m, 2H, OCH₂), 1.42 (s, 2H, NH₂); MS (APCI+) m/z 584.2, 582.2 (M+1).

Procedure B. (3-Chloro-4-fluoro-phenyl)-(3,4-dimethoxy-benzyl)-[7-(3-morpholin-4-yl-propoxy)-6-nitro-quinazolin-4-yl]-amine (44.3 g, 72.5 mmol) and 1% Pt/C (15.0 g; dry wt. 5.48 g) were charged to a pressure reactor. THF (275 mL) was added and the mixture hydrogenated at 3.48 bar and 70° C. until consumption of all of the starting material (about 10 h). The reaction mixture was filtered through Celite and the cake washed with THF (2×50 mL). The resulting solution was reduced in vacuo to approximately 100 mL total volume and distilled with THF (3×100 mL) to remove water from the reaction mixture. The resulting solution of N4-(3-chloro-4-fluoro-phenyl)-N4-(3,4-dimethoxybenzyl)-7-(3-morpholin-4-yl-propoxy)-quinazoline-4,6-diamine in THF (100 mL) was used in subsequent transformations without isolation. HPLC purity: 97.2% (area %).

### Example 6

### Preparation of N-[4-[(3-chloro-4-fluoro-phenyl)-(3,4-dimethoxy-benzyl)-amino]-7-(3-morpholin-4-yl-propoxy)-quinazolin-6-yl]-acrylamide

Procedure A. N4-(3-chloro-4-fluoro-phenyl)-N4-(3,4-dimethoxy-benzyl)-7-(3-morpholin-4-yl-propoxy)-quinazoline-4,6-diamine (0.10 g, 0.17 mmol) was dissolved in ethyl acetate (1.5 mL) and triethyl amine (113 μL, 0.81 mmol) was added. The resulting solution was cooled to −5.0° C. in an ice-salt bath. A solution of acryloyl chloride (18 μL, 0.21 mmol) in ethyl acetate (525 μL) was added with stirring over a period of 10 min. The reaction mixture was allowed to gradually warm to room temperature over a 2 h period, during which time it turned from a very pale yellow to a cloudy yellow-orange in color. The mixture was allowed to stir for 12 h followed by the addition of more acryloyl chloride (25 μL, 0.29 mmol) in portions at room temperature over a 9 h period. The reaction mixture was again stirred for 12 h at room temperature. Saturated sodium chloride (10 mL) was then added with stirring, and the reaction mixture separated into a clear aqueous layer and a yellow organic layer. The aqueous layer was twice extracted with ethyl acetate. The pooled organic phases were dried over magnesium sulfate, filtered and evaporated under reduced pressure to give a waxy yellow glass (0.091 g) which was dissolved in a small amount of methylene chloride, placed on top of a BIOTAGE 12M cartridge and chromatographed, eluting with 5 to 10% isopropanol in methylene chloride. Product-containing fractions were pooled together and evaporated under reduced pressure to give a yellow foam. Diethyl ether was added to this foam and the mixture was ultrasonicated to give a yellow solid that was filtered and dried to give N-[4-[(3-chloro-4-fluoro-phenyl)-(3,4-dimethoxy-benzyl)-amino]-7-(3-morpholin-4-yl-propoxy)-quinazolin-6-yl]-acrylamide (0.044 g, 40%). ¹H NMR (400 MHz, CDCl₃): δ 8.68 (s, 1H, H-2), 8.31 (s, 1H), 7.91 (bs, 1H, NH), 7.17 (s, 1H), 7.03 (m, 2H), 6.91 (m, 2H), 6.78 (m, 1H), 6.67 (m, 1H), 6.25 (s, 2H, CH═CH₂), 5.66 (m, 1H, CH═CH₂), 5.29 (s, 2H, BnCH₂N), 4.19 (t, 2H, J=6.35 Hz, OCH₂), 3.77 (s, 3H, OCH₃), 3.73 (bs, 4H, CH₂OCH₂), 3.72 (s, 3H, OCH₃), 2.56 (bd, 6H, NCH₂), 2.11 (bs, 2H, CH₂CH₂CH₂); ¹⁹F NMR (376.3 MHz, CDCl₃): δ −117.88; MS (APCI+) m/z 638.2, 636.2 (M+1).

Procedure B. A solution of N4-(3-chloro-4-fluoro-phenyl)-N4-(3,4-dimethoxy-benzyl)-7-(3-morpholin-4-yl-propoxy)-quinazoline-4,6-diamine in THF (100 mL; 0.72M; approx. 72.5 mmol) was charged to a flask, diluted with THF (100 mL) and cooled to 0-5° C. Triethylamine (8.44 g, 83.4 mmol) was charged followed by acryloyl chloride (7.55 g, 83.4 mmol) drop wise over 15 min, while maintaining the temperature of the reaction mixture at <10° C. during the course of addition. Upon completion of the reaction (approx. 30 min) the reaction mixture was quenched by addition of an aqueous mixture of NaOH and NaCl (100 mL, 1.2 M in NaOH). After stirring and warming to ambient temperature, the phases were separated and the organic layer washed with brine (50 mL). The organic phase containing N-[4-[(3-chloro-4-fluoro-phenyl)-(3,4-dimethoxy-benzyl)-amino]-7-(3-morpholin-4-yl-propoxy)-quinazolin-6-yl]-acrylamide was reduced in volume to approximately 100 mL and used without purification in subsequent transformations.

### Example 7

### Preparation of N-[4-(3-chloro-4-fluoro-phenyl-amino)-7-(3-morpholin-4-yl-propoxy)-quinazolin-6-yl]-acrylamide

Procedure A. N-[4-[(3-chloro-4-fluoro-phenyl)-(3,4-dimethoxy-benzyl)-amino]-7-(3-morpholin-4-yl-propoxy)-quinazolin-6-yl]-acrylamide (0.044 g, 0.069 mmol) was dissolved in trifluoroacetic acid (4 mL) then heated to reflux for 6 h. After cooling, the TFA was evaporated under reduced pressure and ethyl acetate was added. The resulting mixture was washed with dilute aqueous sodium bicarbonate. The organic layer was then washed with brine, dried over magnesium sulfate, filtered and evaporated under reduced pressure to give the crude product as a yellow foam. Chromatography on a BIOTAGE 12S cartridge eluted with 10% isopropanol in methylene chloride gave N-[4-(3-chloro-4-fluoro-phenyl-amino)-7-(3-morpholin-4-yl-propoxy)-quinazolin-6-yl]-acrylamide (0.031 g, 92.9%) as a white solid. ¹H NMR (400 MHz, DMSO-d₆): δ 9.87 (s, 1H, CONH), 9.67 (s, 1H, NH), 8.93 (s, 1H, H-5), 8.60 (s, 1H, H-2), 8.20 (dd, J=7.05, 2.5 Hz, 1H, H-2′), 7.89-7.85 (m, 1H, H-6′), 7.49 (t, J=9.0 Hz, 1H, H-5′), 7.36 (s, 1H, H-8), 6.78 (dd, J=10.2, 17.1 Hz, 1H, CH═CH₂), 6.38 (dd, J=1.9, 17.1 Hz, 1H, CH═CH₂), 5.88 (dd, J=1.9, 10.2 Hz, 1H, CH═CH₂), 4.34 (t, J=6.3 Hz, 2H, CH₂O), 3.66 (bs, 4H, —CH₂NCH₂—), 2.51 (bs, 4H, —CH₂OCH₂—), 2.08 (bt, J=6.6 Hz, 2H, CH₂CH₂CH₂); MS (APCI+) m/z 486.2, 488.2 (M+1).

Procedure B. A THF solution (50 mL approx. 0.72 M, 36.2 mmol) of N-[4-[(3-chloro-4-fluoro-phenyl)-(3,4-dimethoxy-benzyl)-amino]-7-(3-morpholin-4-yl-propoxy)-quinazolin-6 yl]-acrylamide was reduced in volume (to approx. 30 mL) under vacuum and cooled to 0-5° C. Methanesulfonic acid (46 mL, 710 mmol) was added, neat, while maintaining the temperature of the reaction mixture at <15° C. The reaction mixture was reduced in volume under vacuum at ambient temperature to approx. 60 mL and stirred at ambient temperature until all starting material was consumed (about 4-5 h). The crude product was obtained by quenching the mixture into NaOH/NaCl aq (300 mL, 3.0 M in NaOH) and filtering the resulting precipitate. The crude product was washed with water (4×25 mL) and dried overnight under a stream of N₂. The crude product was recrystallized from acetone to yield N-[4-[(3-chloro-4-fluoro-phenylamino)-7-(3-morpholin-4-yl-propoxy)-quinazolin-6 yl]-acrylamide (13.00 g, 26.88 mmol, 73.9% from (3-chloro-4-fluoro-phenyl)-(3,4-dimethoxy-benzyl)-[7-(3-morpholin-4-yl-propoxy)-6-nitro-quinazolin-4-yl]-amine) as a slightly yellow solid.

### Example 8

### Preparation of (3-chloro-4-fluorophenyl)-[7-(3-morpholin-4-yl-propoxy)-6-nitroquinazolin-4-yl]-amine

One hundred fifty g of 7-fluoro-6-nitroquinazolin-4(3H)-one was suspended in 600 mL of SOCl₂ and, after addition of 6 mL of DMF, boiled under reflux for about 24 h, resulting in a clear solution. About 350 mL of SOCl₂ was distilled off under vacuum, and the resulting coarse crystalline suspension was admixed with about 600 mL of toluene. Approximately 800 mL of the resultant solution was distilled off under vacuum. The distillation was repeated three times with, in each case, 600 mL of fresh toluene. In the last distillation, most of the toluene was distilled off, resulting in a coarse crystalline suspension. The nearly dry residue was mixed with 1.2 L of a THF/t-BuOH mixture (7:3 v/v), and the resulting suspension was cooled to about 10° C. A solution of 114 g of 3-chloro-4-fluoroaniline and 258 g of 3-morpholin-4-yl-propan-1-ol in 300 mL of a THF/ t-BuOH mixture (7:3 v/v) was added drop-wise with stirring and cooling over the course of about 20 min so that the temperature in the reactor remained at a temperature between 10° C. and 15° C. The initially yellowish suspension became less viscous and changed to a yellow-orange color during addition of the 3-chloro-4-fluoroaniline and 3-morpholin-4-yl-propan-1-ol solution. The resulting reaction mixture was allowed to slowly reach room temperature and was subsequently stirred at room temperature for at least 24 h.

With stirring and cooling, a solution of 324 g of t-BuOK in 1.86 L of THF was added drop-wise to the yellow-orange suspension over the course of about 20 min so that the temperature in the reactor remained between 15° C. and 20° C. After the addition of about one-third of the t-BuOK/THF solution, the reaction mixture turned dark red. Once all of the t-BuOK/THF solution was added, the reaction mixture was stirred for an additional 30 min and subsequently stirred into a mixture of 5.4 kg of ice, 6.0 L EtOH and 1.8 L HCl (pH of the solution about 8). The reaction mixture was initially yellow-orange, but after brief stirring, a yellow product crystallized out. The resulting suspension was stirred for about 5 h at about 0° C. and subsequently filtered off with suction. The filter cake was washed twice with 500 mL aliquots of ice-cold EtOH. The product of the one-pot synthesis, (3-chloro-4-fluorophenyl)-[7-(3-morpholin-4-yl-propoxy)-6-nitroquinazolin-4-yl]-amine, was initially dried in a circulating air cabinet at 40° C. and was subsequently dried at 60° C. to constant weight (yield: 316.5 g=95.5%; HPLC purity: 98.48 rel. %; H₂O (by K. F.) 3.69%; mp 257° C.).

### Example 9

### Preparation of N-(3-chloro-4-fluoro-phenyl)-N-[7-(3-morpholin-4-yl-propoxy)-6-nitro-quinazolin-4-yl]-acetamide

Procedure A. A suspension of 10 g of (3-chloro-4-fluoro-phenyl)-[7-(3-morpholin-4-yl-propoxy)-6-nitro-quinazolin-4-yl]-amine and 14 g of cesium carbonate in 150 mL of dry acetonitrile was vigorously stirred at room temperature for 15 min. After cooling to 0° C. and further stirring for 15 min, a solution of 2 g of acetyl chloride in 20 mL of acetonitrile was added drop-wise over 20 min. After stirring for 15 min, the beige-colored suspension was poured into 500 mL of an ice/water mixture. The beige precipitate was filtered off by suction, washed three times with 50 mL of water each and dried in a circulating air drier at 80° C. to furnish 10.0 g of N-(3-chloro-4-fluoro-phenyl)-N-[7-(3-morpholin-4-yl-propoxy)-6-nitro-quinazolin-4-yl]-acetamide (mp 154° C., MS: MG 503).

Procedure B. A suspension of 10.5 grams of (3-chloro-4-fluoro-phenyl)-[7-(3-morpholin-4-yl-propoxy)-6-nitro-quinazolin-4-yl]-amine and 76 mL of acetic anhydride was stirred and heated to 90° C. for about 12-18 hours. The reaction mixture was cooled and distilled under vacuum to remove about 60 mL of acetic anhydride and cooled to 35±5° C. To the resulting slurry was charged 10 mL heptane followed by 33 mL MTBE and stirred at 0-5° C. The product was filtered and washed with cold MTBE and dried in a vacuum oven at 45-50° C. to yield 9.85 g (86% yield) of N-(3-chloro-4-fluoro-phenyl)-N-[7-(3-morpholin-4-yl-propoxy)-6-nitro-quinazolin-4-yl]-acetamide.

### Example 10

### Preparation of N-[6-amino-7-(3-morpholin-4-yl-propoxy)-quinazolin-4-yl]-N-(3-chloro-4-fluoro-phenyl)-acetamide

A mixture of 29.4 g of N-(3-chloro-4-fluoro-phenyl)-N-[7-(3-morpholin-4-yl-propoxy)-6-nitro-quinazolin-4-yl]-acetamide, 7.8 g of 1% platinum on carbon catalyst (JM 156) and 150 mL of MeOH/THF (2:1 v/v) was hydrogenated at 10 bar hydrogen and 90° C. for 4 h. After cooling to room temperature, the catalyst was filtered off and the filtrate evaporated in vacuo to give a reddish oil. The residue was chromatographed in portions on silica gel (0.040-0.063 mesh) with CH₂Cl₂/MeOH (10:1 v/v) as eluent. The product-containing fractions were combined, filtered and evaporated in vacuo to furnish 27.3 g of N-[6-amino-7-(3-morpholin-4-yl-propoxy)-quinazolin-4-yl]-N-(3-chloro-4-fluoro-phenyl)-acetamide as reddish oil (MS: MG 473).

### Example 11

### Preparation of N-[4-[acetyl-(3-chloro-4-fluoro-phenyl)-amino]-7-(3-morpholin-4-yl-propoxy)-quinazolin-6-yl]-acrylamide

To a solution of 27 g of N-[6-amino-7-(3-morpholin-4-yl-propoxy)-quinazolin-4-yl]-N-(3-chloro-4-fluoro-phenyl)-acetamide and 29.5 g of triethylamine in 407 mL of dry THF was added drop-wise with stirring at −5 to −2° C. a solution of 5.7 g of acryloyl chloride in 60 mL of THF over 30 min. After stirring for 1 h at 0° C., the reaction mixture was filtered through a Buchner funnel (Por 3), the filtrate was evaporated in vacuo, and the residue dissolved in 400 mL of ethyl acetate. The organic solution was washed with 200 mL of brine and then dried over sodium sulfate. After filtration and concentration in vacuo, the product crystallized from ethyl acetate to give 19.2 g of beige product, N-[4-[acetyl-(3-chloro-4-fluoro-phenyl)-amino]-7-(3-morpholin-4-yl-propoxy)-quinazolin-6-yl]-acrylamide (mp 143° C.; MS: MG 527; elemental analysis: C 59.15, H 5.15, N 13.26, Cl 6.71, F 3.60 (calculated), C 58.96, H 5.47, N 13.13, Cl 6.84, F 3.47 (measured)).

### Example 12

### Preparation of N-[4-(3-chloro-4-fluoro-phenyl-amino)-7-(3-morpholin-4-yl-propoxy)-quinazolin-6-yl]-acrylamide

A solution of 5 g of N-[4-[acetyl-(3-chloro-4-fluoro-phenyl)-amino]-7-(3-morpholin-4-yl-propoxy)-quinazolin-6-yl]-acrylamide in 100 mL aq 1 N HCl is stirred at room temperature for 4 days. The greenish precipitate is filtered off and dried in a circulating air drier at 60° C. to constant weight to give 3.3 g of a hydrochloride salt of N-[4-(3-chloro-4-fluoro-phenyl-amino)-7-(3-morpholin-4-yl-propoxy)-quinazolin-6-yl]-acrylamide.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference in their entirety and for all purposes.

What is claimed is:

1. A method of making a compound of Formula 1, 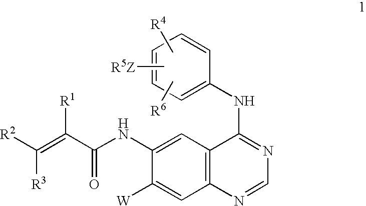
or a pharmaceutically acceptable salt, ester, amide or prodrug thereof, in which
R¹, R² and R³ are independently hydrogen, halogen, NO₂, CN, CF₃, C₁₋₆ alkyl, C₁₋₆ haloalkyl, C₂₋₆ alkenyl, C₂₋₆ alkynyl, C₃₋₈ cycloalkyl, C₃₋₈ heterocyclyl, carboxy, C₁₋₆ alkoxycarbonyl, C₁₋₆ alkylcarbamoyl, aryl-(CH₂)ₘ, heteroaryl-(CH₂)ₘ, heterocyclyl-(CH₂)ₘ, (CH₂)ₘCO₂R⁸, (CH₂)ₘS(O)ₙR⁸, (CH₂)ₘSO₂NR⁸R⁹, OR⁸, SR⁸, (CH₂)ₘNR⁸R⁹, (CH₂)ₘN(O)R⁸R⁹, (CH₂)ₘP(O)(OR⁸)(OR⁹), (CH₂)ₘCOR⁸, (CH₂)ₘCO₂R⁸, (CH₂)ₘC(O)NR⁸R⁹, (CH₂)ₘC(O)NR⁸SO₂R⁸, (CH₂)ₘNR⁸SO₂R⁹, (CH₂)ₘC(O)NR⁸OR⁹, (CH₂)ₘS(O)ₙR⁸, or (CH₂)ₘSO₂NR⁸R⁹, wherein aryl-(CH₂)ₘ includes phenylalkyl or substituted phenylalkyl having from one to three substituents that are independently NO₂, CN, CF₃, C₁₋₆ alkyl-NH, (C₁₋₆ alkyl)₂N, or monocyclic heteroaryl, and each C₁₋₆ alkyl is optionally substituted with OH, NH₂ or —N(A)B;
R⁴ and R⁶ are independently hydrogen, hydroxy, halogen, C₁₋₄ alkyl, C₁₋₄ alkoxy, C₁₋₄ alkylamino, C₁₋₄ alkyldiamino, C₁₋₄ alkylthio, C₁₋₄ alkylsulfinyl, C₁₋₄ alkylsulfonyl, C₁₋₄ alkylcarbonyl, C₁₋₄ alkylcarbamoyl, dicarbamoyl, carbamyl, C₁₋₄ alkoxycarbonyl, cyano, nitro, or trifluoromethyl;
R⁵ is phenyl, pyridyl, furyl, thiazolyl, imidazolyl or thienyl, each optionally having one or two substituents that are independently halogen, C₁₋₆ alkyl, C₁₋₆ alkoxy, hydroxy, amino, cyano, C₁₋₆ alkyl-NH or (C₁₋₆ alkyl)₂N;
W is SR⁷, OR⁷ or NHR⁷; and
Z is hydrogen, halogen, C₁₋₆ alkyl, C₃₋₈ cycloalkyl, C₁₋₆ alkoxy, C₃₋₈ cycloalkoxy, nitro, C₁₋₆ haloalkyl, hydroxy, C₁₋₆ acyloxy, NH₂, C₁₋₆ alkyl-NH, (C₁₋₆ alkyl)₂N, C₃₋₈ cycloalkyl-NH, (C₃₋₈ cycloalkyl)₂N, hydroxymethyl, C₁₋₆ alkylcarbonyl, cyano, azido, C₁₋₆ thioalkyl, C₁₋₆ sulfinylalkyl, C₁₋₆ sulfonylalkyl, C₃₋₈ thiocycloalkyl, C₃₋₈ sulfinylcycloalkyl, C₃₋₈ sulfonylcycloalkyl, mercapto, C₁₋₆ alkoxycarbonyl, C₃₋₈ cycloalkoxycarbonyl, C₂₋₄ alkenyl, C₄₋₈ cycloalkenyl, or C₂₋₄ alkynyl, provided that when Z is monovalent, R⁵ is absent;
wherein, R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
R⁸ and R⁹ are each independently hydrogen, C₁₋₆ alkyl, C₁₋₆ haloalkyl, C₂₋₆ alkenyl, C₂₋₆ alkynyl, arylalkyl, cycloalkyl, heterocyclyl, aryl, heteroaryl, or heteroarylalkyl;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
n and m are, respectively, integers from zero to two, inclusive, and from zero to four,
inclusive;
the method comprising:
removing a protecting group, G, from a compound of Formula 10, 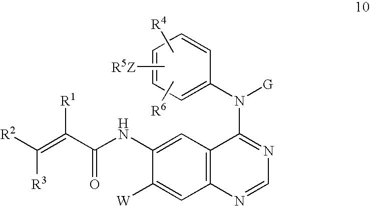
to yield the compound of Formula 1; and
optionally converting the compound of Formula 1 to a pharmaceutically acceptable salt, ester, amide or prodrug thereof.

2. The method of claim 1, further comprising reacting a compound of Formula 7, 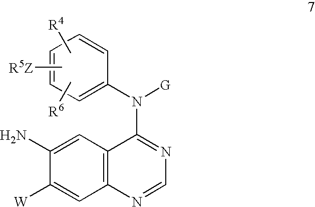
with a compound of Formula 8, 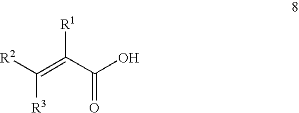
or with a compound of Formula 9, 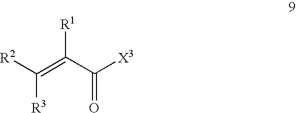
to yield the compound of Formula 10, wherein G, R¹, R², R³, R⁴, R⁵, R⁶, W, and Z are as defined in claim 1, X³ is a leaving group, and provided that when G is Boc, W is not alkoxy.

3. The method of claim 2, further comprising reacting a compound of Formula 6, 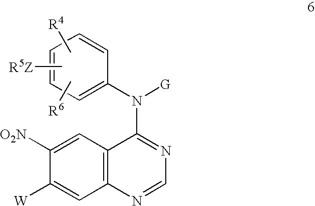
with hydrogen in the presence of a catalyst or with a reducing agent to yield the compound of claim 7, wherein G, R⁴, R⁵, R⁶, W, and Z are as defined in claim 1.

4. The method of claim 3, further comprising installing the protecting group, G, on a compound of Formula 5, 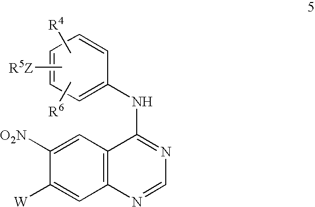
to yield the compound of Formula 6, wherein G, R⁴, R⁵, R⁶, W, and Z are as defined in claim 1.

5. The method of claim 3, further comprising displacing a leaving group, X², of Formula 12, 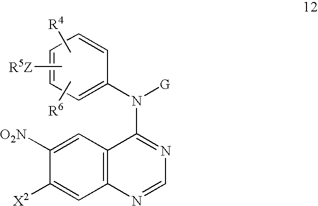
with W to yield the compound of Formula 6, wherein G, R⁴, R⁵, R⁶, W, and Z are as defined in claim 1, and provided that when G is Boc, X² is not halogen.

6. The method of claim 5, further comprising reacting a compound of Formula 2, 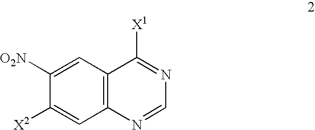
with a compound of Formula 11, 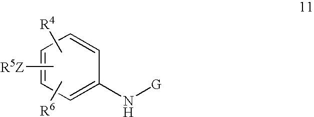
to yield the compound of Formula 12, wherein G, R⁴, R⁵, R⁶, and Z are as defined in claim 1, X² is as defined in claim 5, and Xi is a leaving group.

7. The method of claim 1, wherein G is acetyl.

8. The method of claim 1, wherein G is dimethoxy benzyl.

9. The method of claim 1, wherein R¹, R², R³ and Z are each hydrogen, and R⁴ and R⁶ are each halogen.

10. The method of claim 1, wherein W is morpholin-4-yl-alkoxy.

11. The method of claim 1, wherein the compound of Formula 1 is N-[4-(3-chloro-4-fluoro-phenylamino)-7-(3-morpholin-4-yl-propoxy)-quinazolin-6-yl]-acrylamide.

12. A method of making a compound of Formula 23, 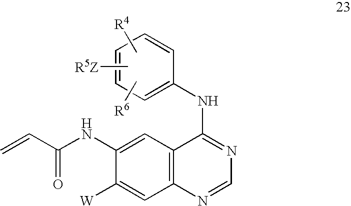
or a pharmaceutically acceptable salt, ester, amide, or prodrug thereof, in which
R⁴ and R⁶ are independently hydrogen, hydroxy, halogen, C₁₋₄ alkyl, C₁₋₄ alkoxy, C₁₋₄ alkylamino, C₁₋₄ alkyldiamino, C₁₋₄ alkylthio, C₁₋₄ alkylsulfinyl, C₁₋₄ alkylsulfonyl, C₁₋₄ alkylcarbonyl, C₁₋₄ alkylcarbamoyl, dicarbamoyl, carbamyl, C₁₋₄ alkoxycarbonyl, cyano, nitro, or trifluoromethyl;
R⁵ is phenyl, pyridyl, furyl, thiazolyl, imidazolyl or thienyl, each optionally having one or two substituents that are independently halogen, C₁₋₆ alkyl, C₁₋₆ alkoxy, hydroxy, amino, cyano, C₁₋₆ alkyl-NH or (C₁₋₆ alkyl)₂N;
W is SR⁷, OR⁷ or NHR⁷; and
Z is hydrogen, halogen, C₁₋₆ alkyl, C₃₋₈ cycloalkyl, C₁₋₆ alkoxy, C₃₋₈ cycloalkoxy, nitro, C₁₋₆ haloalkyl, hydroxy, C₁₋₆ acyloxy, NH₂, C₁₋₆ alkyl-NH, (C₁₋₆ alkyl)₂N, C₃₋₈ cycloalkyl-NH, (C₃₋₈ cycloalkyl)₂N, hydroxymethyl, C₁₋₆ alkylcarbonyl, cyano, azido, C₁₋₆ thioalkyl, C₁₋₆ sulfinylalkyl, C₁₋₆ sulfonylalkyl, C₃₋₈ thiocycloalkyl, C₃₋₈ sulfinylcycloalkyl, C₃₋₈ sulfonylcycloalkyl, mercapto, C₁₋₆ alkoxycarbonyl, C₃₋₈ cycloalkoxycarbonyl, C₂₋₄ alkenyl, C₄₋₈ cycloalkenyl, or C₂₋₄ alkynyl, provided that when Z is monovalent, R⁵ is absent;
wherein, R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
m is an integer from zero to four, inclusive;
the method comprising:
eliminating SR¹² from a compound of Formula 22, 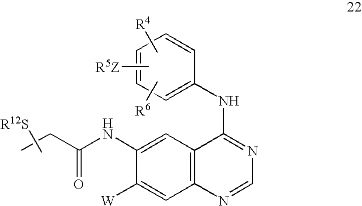
to yield the compound of Formula 23; and
optionally converting the compound of Formula 23 to a pharmaceutically acceptable salt, ester, amide, or prodrug thereof, wherein R¹² is C₁₋₆ alkyl or aryl.

13. The method of claim 12, further comprising reacting a compound of Formula 21, 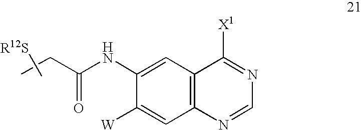
with a compound of Formula 3, 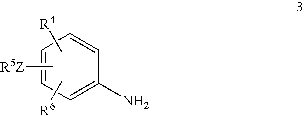
to yield the compound of Formula 22, wherein R⁴, R⁵, R⁶, R¹², W, and Z are as defined in claim 12, and X¹ is a leaving group.

14. The method of claim 13, further comprising reacting a compound of Formula 18, 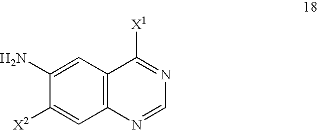
with a compound of Formula 19, 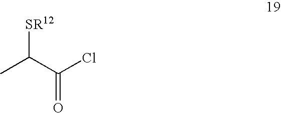
or with a compound of Formula 20, 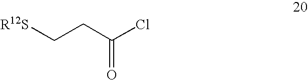
to yield the compound of Formula 21, wherein R¹² and W are as defined in claim 12, and X¹ is as defined in claim 13.

15. The method of claim 12, wherein Z is hydrogen, and R⁴ and R⁶ are each halogen.

16. The method of claim 12, wherein W is morpholin-4-yl-alkoxy.

17. The method of claim 12, wherein the compound of Formula 23 is N-[4-(3-chloro-4-fluoro-phenylamino)-7-(3-morpholin-4-yl-propoxy)-quinazolin-6-yl]-acrylamide.

18. A method of making a compound of Formula 29, 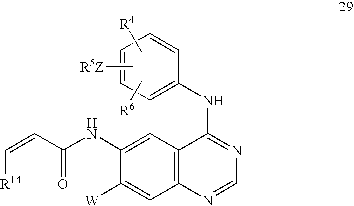
or a pharmaceutically acceptable salt, ester, amide, or prodrug thereof, in which
R⁴ and R⁶ are independently hydrogen, hydroxy, halogen, C₁₋₄ alkyl, C₁₋₄ alkoxy, C₁₋₄ alkylamino, C₁₋₄ alkyldiamino, C₁₋₄ alkylthio, C₁₋₄ alkylsulfinyl, C₁₋₄ alkylsulfonyl, C₁₋₄ alkylcarbonyl, C₁₋₄ alkylcarbamoyl, dicarbamoyl, carbamyl, C₁₋₄ alkoxycarbonyl, cyano, nitro, or trifluoromethyl;
R⁵ is phenyl, pyridyl, furyl, thiazolyl, imidazolyl or thienyl, each optionally having one or two substituents that are independently halogen, C₁₋₆ alkyl, C₁₋₆ alkoxy, hydroxy, amino, cyano, C₁₋₆ alkyl-NH or (C₁₋₆ alkyl)₂N;
W is SR⁷, OR⁷ or NHR⁷;
Z is hydrogen, halogen, C₁₋₆ alkyl, C₃₋₈ cycloalkyl, C₁₋₆ alkoxy, C₃₋₈ cycloalkoxy, nitro, C₁₋₆ haloalkyl, hydroxy, C₁₋₆ acyloxy, NH₂, C₁₋₆ alkyl-NH, (C₁₋₆ alkyl)₂N, C₃₋₈ cycloalkyl-NH, (C₃₋₈ cycloalkyl)₂N, hydroxymethyl, C₁₋₆ alkylcarbonyl, cyano, azido, C₁₋₆ thioalkyl, C₁₋₆ sulfinylalkyl, C₁₋₆ sulfonylalkyl, C₃₋₈ thiocycloalkyl, C₃₋₈ sulfinylcycloalkyl, C₃₋₈ sulfonylcycloalkyl, mercapto, C₁₋₆ alkoxycarbonyl, C₃₋₈ cycloalkoxycarbonyl, C₂₋₄ alkenyl, C₄₋₈ cycloalkenyl, or C₂₋₄ alkynyl, provided that when Z is monovalent, R⁵ is absent; and
R¹⁴ is hydrogen, halogen, C₂₋₆ alkenyl, C₂₋₆ alkynyl, and C₂-6 alkenyl or C₂₋₆ alkynyl substituted with hydroxy, alkoxy, amino or alkylamino;
wherein, R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
m is an integer from zero to four, inclusive;
the method comprising:
removing [1,3,4]oxadiazole from a compound of Formula 28, 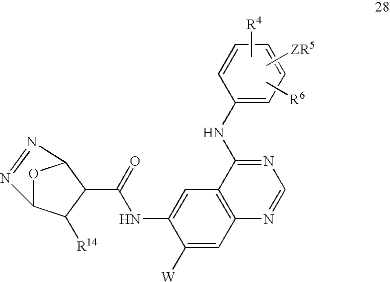
to yield the compound of Formula 29; and
optionally converting the compound of Formula 29 to a pharmaceutically acceptable salt, ester, amide, or prodrug thereof.

19. The method of claim 18, further comprising removing ester moieties, R¹³O₂C, from a compound of Formula 27, 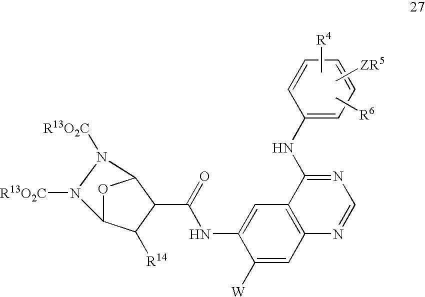
to yield the compound of Formula 28, wherein R⁴, R⁵, R⁶, R¹⁴, W, and Z are as defined in claim 18, and R¹³ is C₁₋₄ alkyl, C₁₋₄ haloalkyl, C₂₋₄ alkenyl, TMS-(CH₂)ₘ or aryl-(CH₂)ₘ.

20. The method of claim 19, further comprising reacting a compound of Formula 26, 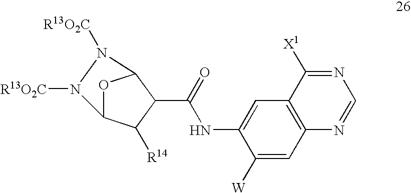
with a compound of Formula 3, 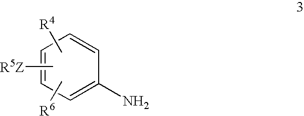
to yield the compound of Formula 27, wherein R⁴, R⁵, R⁶, R¹⁴, W, and Z are as defined in claim 18, R¹³ is as defined in claim 19, and X¹ is a leaving group.

21. The method of claim 20, further comprising reacting a compound of Formula 18, 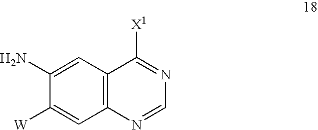
with a compound of Formula 24 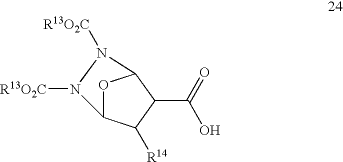
or with a compound of Formula 25 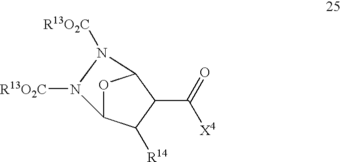
to yield the compound of Formula 26, wherein R¹⁴ and W are as defined in claim 18, R¹³ is as defined in claim 19, X¹ is as defined in claim 20, and X⁴ is a leaving group.

22. The method of claim 19, further comprising reacting a compound of Formula 36, 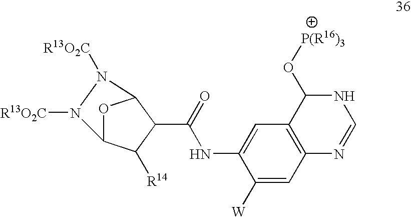
with a compound of Formula 3, 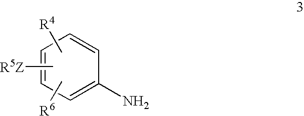
to yield the compound of Formula 27, wherein R⁴, R⁵, R⁶, R¹⁴, W, and Z are as defined in claim 18, R¹³ is as defined in claim 19, and R¹⁶ is C₁₋₆ alkyl, phenyl, or phenoxy.

23. The method of claim 22, further comprising reacting a compound of Formula 34 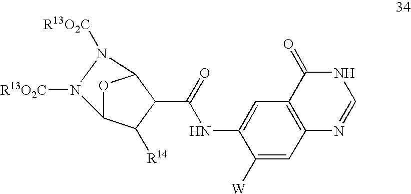
with (R¹⁶)₃P(X⁵)₂ to yield the compound of 36, wherein R¹⁴ and W are as defined in claim 18, R¹³ is as defined in claim 19, R¹⁶ is as defined in claim 22, and X⁵ is hydrogen, halogen or absent.

24. The method of claim 19, further comprising reacting a compound of Formula 34, 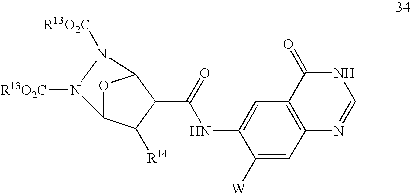
with a compound of Formula 37, 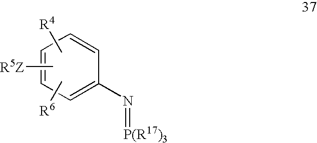
to yield the compound of Formula 27, wherein R⁴, R⁵, R⁶, R¹⁴, W, and Z are as defined in claim 18, R¹³ is as defined in claim 19, and R¹⁷ is C₁₋₆ alkyl, phenyl or phenoxy.

25. The method of claim 24, further comprising reacting a compound of Formula 33, 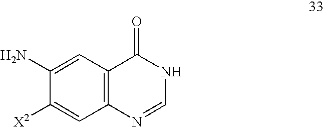
with a compound of Formula 24, 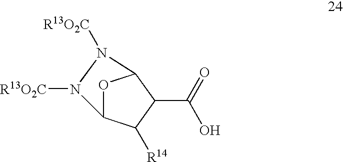
or with a compound of Formula 25, 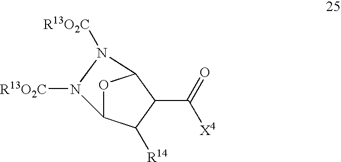
to yield the compound of Formula 34, wherein R¹⁴ and W are as defined in claim 18, R¹³ is as defined in claim 19, and X⁴ is a leaving group.

26. The method of claim 19, further comprising reacting a compound of Formula 38, 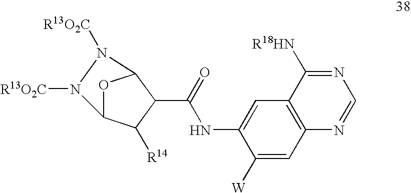
with a compound of Formula 39, 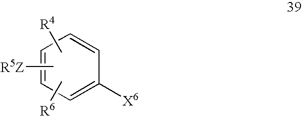
in the presence of a catalyst to yield a compound of Formula 40, 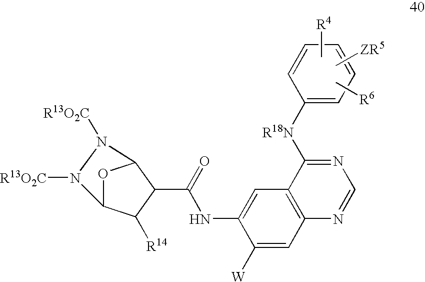
wherein R⁴, R⁵, R⁶, R¹⁴, W, and Z are as defined in claim 18, R¹³ is as defined in claim 19, X⁶ is halogen, and R¹⁸ is hydrogen or a group that facilitates coupling of the compounds of Formula 38 and Formula 39; and
optionally reacting the compound of Formula 40 with an acid to yield the compound of Formula 27 when R¹⁸ is non-hydrogen.

27. The method of claim 18, wherein Z and R¹⁴ are each hydrogen, and R⁴ and R⁶ are each halogen.

28. The method of claim 18, wherein W is morpholin-4-yl-alkoxy.

29. The method of claim 18, wherein the compound of Formula 29 is N-[4-(3-chloro-4-fluoro-phenylamino)-7-(3-morpholin-4-yl-propoxy)-quinazolin-6-yl]-acrylamide.

30. A method of making a compound of Formula 46, 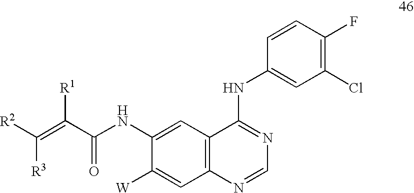
or a pharmaceutically acceptable salt, ester, amide, or prodrug thereof, in which
R¹, R² and R³ are independently hydrogen, halogen, NO₂, CN, CF₃, C₁₋₆ alkyl, C₁₋₆ haloalkyl, C₂₋₆ alkenyl, C₂₋₆ alkynyl, C₃₋₈ cycloalkyl, C₃₋₈ heterocyclyl, carboxy, C₁₋₆ alkoxycarbonyl, C₁₋₆ alkylcarbamoyl, aryl-(CH₂)ₘ, heteroaryl-(CH₂)ₘ, heterocyclyl-(CH₂)ₘ, (CH₂)ₘCO₂R⁸, (CH₂)ₘS(O)ₙR⁸, (CH₂)ₘSO₂NR⁸R⁹, OR⁸, SR⁸, (CH₂)ₘNR⁸R⁹, (CH₂)ₘN(O)R⁸R⁹, (CH₂)ₘP(O)(OR⁸)(OR⁹), (CH₂)ₘCOR⁸, (CH₂)ₘCO₂R⁸, (CH₂)ₘC(O)NR⁸R⁹, (CH₂)ₘC(O)NR⁸SO₂R⁸, (CH₂)ₘNR⁸SO₂R⁹, (CH₂)ₘC(O)NR⁸OR⁹, (CH₂)ₘS(O)ₙR⁸, or (CH₂)ₘSO₂NR⁸R⁹, wherein aryl-(CH₂)ₘ includes phenylalkyl or substituted phenylalkyl having from one to three substituents that are independently NO₂, CN, CF₃, C₁₋₆ alkyl-NH, (C₁₋₆ alkyl)₂N, or monocyclic heteroaryl, and each C₁₋₆ alkyl is optionally substituted with OH, NH₂ or —N(A)B; and
W is SR⁷, OR⁷ or NHR⁷;
wherein, R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
R⁸ and R⁹ are each independently hydrogen, C₁₋₆ alkyl, C₁₋₆ haloalkyl, C₂₋₆ alkenyl, C₂₋₆ alkynyl, arylalkyl, cycloalkyl, heterocyclyl, aryl, heteroaryl, or heteroarylalkyl;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
n and m are, respectively, integers from zero to two, inclusive, and from zero to four,
inclusive;
the method comprising:
treating a compound of Formula 45, 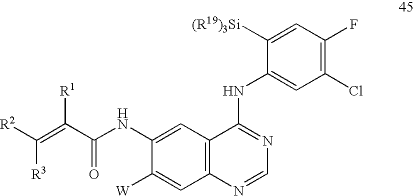
with an acid to yield the compound of Formula 46, wherein R¹⁹ is C₁₋₄ alkyl, C₁₋₄ alkoxy or aryl; and
optionally converting the compound of Formula 46 to a pharmaceutically acceptable salt, ester, amide, or prodrug thereof.

31. The method of claim 30, wherein R¹, R² and R³ are each hydrogen.

32. The method of claim 30, wherein W is morpholin-4-yl-alkoxy.

33. The method of claim 30, wherein the compound of Formula 46 is N-[4-(3-chloro-4-fluoro-phenylamino)-7-(3-morpholin-4-yl-propoxy)-quinazolin-6-yl]-acrylamide.

34. A compound of Formula 47, 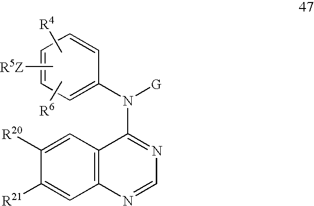
or a pharmaceutically acceptable salt, ester, amide or prodrug thereof, in which
R⁴ and R⁶ are independently hydrogen, hydroxy, halogen, C₁₋₄ alkyl, C₁₋₄ alkoxy, C₁₋₄ alkylamino, C₁₋₄ alkyldiamino, C₁₋₄ alkylthio, C₁₋₄ alkylsulfinyl, C₁₋₄ alkylsulfonyl, C₁₋₄ alkylcarbonyl, C₁₋₄ alkylcarbamoyl, dicarbamoyl, carbamyl, C₁₋₄ alkoxycarbonyl, cyano, nitro, or trifluoromethyl;
R⁵ is phenyl, pyridyl, furyl, thiazolyl, imidazolyl or thienyl, each optionally having one or two substituents that are independently halogen, C₁₋₆ alkyl, C₁₋₆ alkoxy, hydroxy, amino, cyano, C₁₋₆ alkyl-NH or (C₁₋₆ alkyl)₂N;
R²⁰ is NH₂, NO₂, or 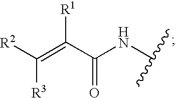
R²¹ is SR⁷, OR⁷, NHR⁷ or a leaving group;
Z is hydrogen, halogen, C₁₋₆ alkyl, C₃₋₈ cycloalkyl, C₁₋₆ alkoxy, C₃₋₈ cycloalkoxy, nitro, C₁₋₆ haloalkyl, hydroxy, C₁₋₆ acyloxy, NH₂, C₁₋₆ alkyl-NH, (C₁₋₆ alkyl)₂N, C₃₋₈ cycloalkyl-NH, (C₃₋₈ cycloalkyl)₂N, hydroxymethyl, C₁₋₆ alkylcarbonyl, cyano, azido, C₁₋₆ thioalkyl, C₁₋₆ sulfinylalkyl, C₁₋₆ sulfonylalkyl, C₃₋₈ thiocycloalkyl, C₃₋₈ sulfinylcycloalkyl, C₃₋₈ sulfonylcycloalkyl, mercapto, C₁₋₆ alkoxycarbonyl, C₃₋₈ cycloalkoxycarbonyl, C₂₋₄ alkenyl, C₄₋₈ cycloalkenyl, or C₂₋₄ alkynyl, provided that when Z is monovalent, R⁵ is absent; and
G is a protecting group, provided that when G is Boc and R²⁰ is NH₂ or NO₂, R²¹ is not halogen or alkoxy;
wherein R¹, R² and R³ are independently hydrogen, halogen, NO₂, CN, CF₃, C₁₋₆ alkyl, C₁₋₆ haloalkyl, C₂₋₆ alkenyl, C₂₋₆ alkynyl, C₃₋₈ cycloalkyl, C₃₋₈ heterocyclyl, carboxy, C₁₋₆ alkoxycarbonyl, C₁₋₆ alkylcarbamoyl, aryl-(CH₂)ₘ, heteroaryl-(CH₂)ₘ, heterocyclyl-(CH₂)ₘ, (CH₂)ₘCO₂R⁸, (CH₂)ₘS(O)ₙR⁸, (CH₂)ₘSO₂NR⁸R⁹, OR⁸, SR^{8,} (CH₂)ₘNR⁸R⁹, (CH₂)ₘN(O)R⁸R⁹, (CH₂)ₘP(O)(OR⁸)(OR⁹), (CH₂)ₘCOR⁸, (CH₂)ₘCO₂R⁸, (CH₂)ₘC(O)NR⁸R⁹, (CH₂)ₘC(O)NR⁸SO₂R⁸, (CH₂)ₘNR⁸SO₂R⁹, (CH₂)ₘC(O)NR⁸OR⁹, (CH₂)ₘS(O)ₙR⁸, or (CH₂)ₘSO₂NR⁸R⁹, wherein aryl-(CH₂)ₘ includes phenylalkyl or substituted phenylalkyl having from one to three substituents that are independently NO₂, CN, CF₃, C₁₋₆ alkyl-NH, (C₁₋₆ alkyl)₂N, or monocyclic heteroaryl, and each C₁₋₆ alkyl is optionally substituted with OH, NH₂ or —N(A)B;
R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
R⁸ and R⁹ are each independently hydrogen, C₁₋₆ alkyl, C₁₋₆ haloalkyl, C₂₋₆ alkenyl, C₂₋₆ alkynyl, arylalkyl, cycloalkyl, heterocyclyl, aryl, heteroaryl, or heteroarylalkyl;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
n and m are, respectively, integers from zero to two, inclusive, and from zero to four, inclusive.

35. The compound of claim 34, wherein G is acetyl.

36. The compound of claim 34, wherein G is dimethoxy benzyl.

37. The compound of claim 34, wherein R²⁰ is NH₂.

38. The compound of claim 37, wherein R²¹ is SR⁷, OR⁷ or NHR₇.

39. The compound of claim 34, wherein R²⁰ is NO₂.

40. The compound of claim 39, wherein R²¹ is SR⁷, OR⁷ or NHR⁷.

41. The compound of claim 34, wherein R²⁰ is

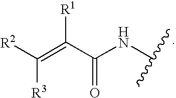

42. The compound of claim 41, wherein R¹, R², R³ and Z are each hydrogen, and R⁴ and R⁶ are each halogen.

43. The compound of claim 34, wherein R²¹ is morpholin-4-yl-alkoxy.

44. A compound selected from:
(3-chloro-4-fluoro-phenyl)-(3,4-dimethoxy-benzyl)-(7-fluoro-6-nitro-quinazolin-4-yl)-amine;
(3-chloro-4-fluoro-phenyl)-(3,4-dimethoxy-benzyl)-[7-(3-morpholin-4-yl-propoxy)-6-nitro-quinazolin-4-yl]-amine;
N4-(3-chloro-4-fluoro-phenyl)-N4-(3,4-dimethoxy-benzyl)-7-(3-morpholin-4-yl-propoxy)-quinazoline-4,6-diamine;
N-[4-[(3-chloro-4-fluoro-phenyl)-(3,4-dimethoxy-benzyl)-amino]-7-(3-morpholin-4-yl-propoxy)-quinazolin-6-yl]-acrylamide;
N-(3-chloro-4-fluoro-phenyl)-N-[7-(3-morpholin-4-yl-propoxy)-6-nitro-quinazolin-4-yl]-acetamide;
N-[6-amino-7-(3-morpholin-4-yl-propoxy)-quinazolin-4-yl]-N-(3-chloro-4-fluoro-phenyl)-acetamide; and
N-[4-[acetyl-(3-chloro-4-fluoro-phenyl)-amino]-7-(3-morpholin-4-yl-propoxy)-quinazolin-6-yl]-acrylamide;
or a pharmaceutically acceptable salt thereof.

45. A compound of Formula 48, 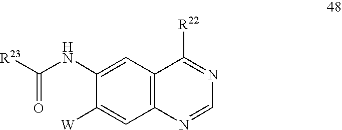
or a pharmaceutically acceptable salt, ester, amide, or prodrug thereof, in which R²² is a leaving group or 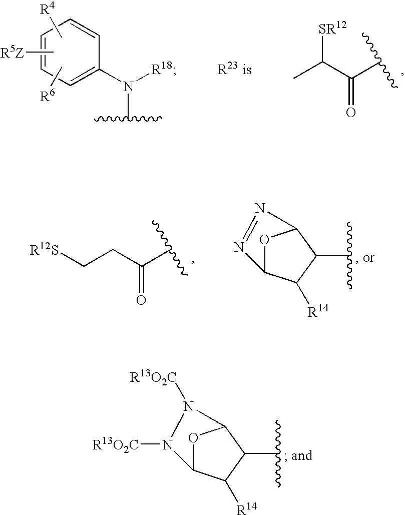
W is SR⁷, OR⁷ or NHR⁷;
wherein R⁴ and R⁶ are independently hydrogen, hydroxy, halogen, C₁₋₄ alkyl, C₁₋₄ alkoxy, C₁₋₄ alkylamino, C₁₋₄ alkyldiamino, C₁₋₄ alkylthio, C₁₋₄ alkylsulfinyl, C₁₋₄ alkylsulfonyl, C₁₋₄ alkylcarbonyl, C₁₋₄ alkylcarbamoyl, dicarbamoyl, carbamyl, C₁₋₄ alkoxycarbonyl, cyano, nitro, or trifluoromethyl;
R⁵ is phenyl, pyridyl, furyl, thiazolyl, imidazolyl or thienyl, each optionally having one or two substituents that are independently halogen, C₁₋₆ alkyl, C₁₋₆ alkoxy, hydroxy, amino, cyano, C₁₋₆ alkyl-NH or (C₁₋₆ alkyl)₂N;
Z is hydrogen, halogen, C₁₋₆ alkyl, C₃₋₈ cycloalkyl, C₁₋₆ alkoxy, C₃₋₈ cycloalkoxy, nitro, C₁₋₆ haloalkyl, hydroxy, C₁₋₆ acyloxy, NH₂, C₁₋₆ alkyl-NH, (C₁₋₆ alkyl)₂N, C₃₋₈ cycloalkyl-NH, (C₃₋₈ cycloalkyl)₂N, hydroxymethyl, C₁₋₆ alkylcarbonyl, cyano, azido, C₁₋₆ thioalkyl, C₁₋₆ sulfinylalkyl, C₁₋₆ sulfonylalkyl, C₃₋₈ thiocycloalkyl, C₃₋₈ sulfinylcycloalkyl, C₃₋₈ sulfonylcycloalkyl, mercapto, C₁₋₆ alkoxycarbonyl, C₃₋₈ cycloalkoxycarbonyl, C₂₋₄ alkenyl, C₄₋₈ cycloalkenyl, or C₂₋₄ alkynyl, provided that when Z is monovalent, R⁵ is absent;
R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
R¹² is C₁₋₆ alkyl or aryl;
R¹³ is C₁₋₄ alkyl, C₁₋₄ haloalkyl, C₂₋₄ alkenyl, TMS-(CH₂)ₘ or aryl-(CH₂)ₘ;
R¹⁴ is hydrogen, halogen, C₂₋₆ alkenyl, C₂₋₆ alkynyl, and C₂₋₆ alkenyl or C₂₋₆ alkynyl substituted with hydroxy, alkoxy, amino or alkylamino;
R¹⁸ is hydrogen, an O-substituted carbonyldioxy radical, or an S-substituted sulfonyl radical, the O-substituted carbonyldioxy radical or the S-substituted sulfonyl radicals independently substituted with t-butyl, allyl, benzyl, p-methoxybenzyl, 2-chloroethyl, 2,2,2-trichloroethyl, 2-trimethylsilylethyl, 2-nitroethyl, 2-cyanoethyl, 4-nitrobenzyl, trifluoroacetyl or Tf;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
m is an integer from zero to four, inclusive.

46. The compound of claim 44, wherein R²² is

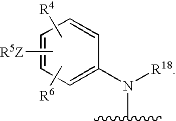

47. The compound of claim 46, wherein R¹⁸ is hydrogen and R²³ is

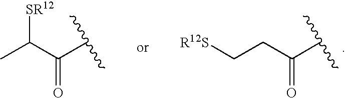

48. The compound of claim 46, wherein R¹⁸ is hydrogen and R²³ is

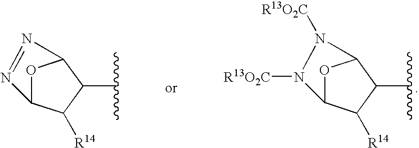

49. The compound of claim 44, wherein R²² is a leaving group.

50. The compound of claim 49, wherein R¹⁸ is hydrogen and R²³ is

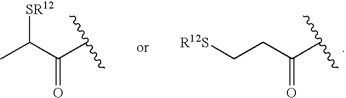

51. The compound of claim 49, wherein R¹⁸ is hydrogen and R²³ is

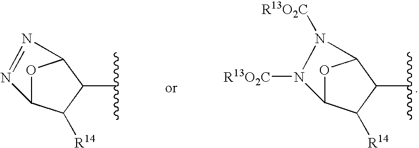

52. A compound of Formula 49, 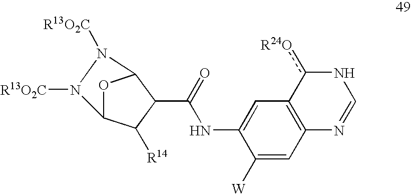
or a pharmaceutically acceptable salt, ester, amide or prodrug thereof, in which
R¹³ is C₁₋₄ alkyl, C₁₋₄ haloalkyl, C₂₋₄ alkenyl, TMS-(CH₂)ₘ or aryl-(CH₂)ₘ;
R¹⁴ is hydrogen, halogen, C₂₋₆ alkenyl, C₂₋₆ alkynyl, and C₂₋₆ alkenyl or C₂₋₆ alkynyl substituted with hydroxy, alkoxy, amino or alkylamino;
R²⁴ is P⁺(R¹⁶)₃ or is absent;
W is SR⁷, R⁷ or NHR⁷;
R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
R¹⁶ is C₁₋₆ alkyl, phenyl, or phenoxy;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
m is an integer from zero to four, inclusive.

53. A compound of Formula 45, 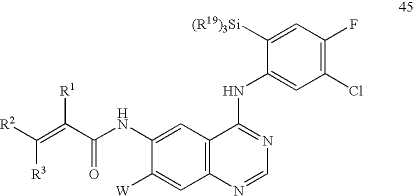
or a pharmaceutically acceptable salt, ester, amide or prodrug thereof, in which
R¹, R² and R³ are independently hydrogen, halogen, NO₂, CN, CF₃, C₁₋₆ alkyl, C₁₋₆ haloalkyl, C₂₋₆ alkenyl, C₂₋₆ alkynyl, C₃₋₈ cycloalkyl, C₃₋₈ heterocyclyl, carboxy, C₁₋₆ alkoxycarbonyl, C₁₋₆ alkylcarbamoyl, aryl-(CH₂)ₘ, heteroaryl-(CH₂)ₘ, heterocyclyl-(CH₂)ₘ, (CH₂)ₘCO₂R⁸, (CH₂)ₘS(O)ₙR⁸, (CH₂)ₘSO₂NR⁸R⁹ OR⁸, SR⁸, (CH₂)ₘNR⁸R⁹, (CH₂)ₘN(O)R⁸R⁹, (CH₂)ₘP(O)(OR⁸)(OR⁹), (CH₂)ₘCOR⁸, (CH₂)ₘCO₂R⁸, (CH₂)ₘC(O)NR⁸R⁹, (CH₂)ₘC(O)NR⁸SO₂R⁸, (CH₂)ₘNR⁸SO₂R⁹, (CH₂)ₘC(O)NR⁸OR⁹, (CH₂)ₘS(O)ₙR⁸, or (CH₂)ₘSO₂NR⁸R⁹, wherein aryl-(CH₂)ₘ includes phenylalkyl or substituted phenylalkyl having from one to three substituents that are independently NO₂, CN, CF₃, C₁₋₆ alkyl-NH, (C₁₋₆ alkyl)₂N, or monocyclic heteroaryl, and each C₁₋₆ alkyl is optionally substituted with OH, NH₂ or —N(A)B;
R¹⁹ is C₁₋₄ alkyl, C₁₋₄ alkoxy or aryl;
W is SR⁷, OR⁷ or NHR⁷; and
wherein, R⁷ is hydrogen, C₁₋₆ alkyl, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, imidazol-1-yl-(CH₂)ₘ, morpholin-4-yl-(CH₂)ₘ, thiomorpholin-4-yl-(CH₂)ₘ, or hexahydroazepin-1-yl-(CH₂)ₘ, wherein each C₁₋₆ alkyl optionally includes one or more substituents that are OH, NH₂ or —N(A)B;
R⁸ and R⁹ are each independently hydrogen, C₁₋₆ alkyl, C₁₋₆ haloalkyl, C₂₋₆ alkenyl, C₂₋₆ alkynyl, arylalkyl, cycloalkyl, heterocyclyl, aryl, heteroaryl, or heteroarylalkyl;
A and B are independently hydrogen, C₁₋₆ alkyl, (CH₂)ₘOH, piperidin-1-yl-(CH₂)ₘ, piperazin-1-yl-(CH₂)ₘ, 4-C₁₋₆ alkyl-piperazin-1-yl-(CH₂)ₘ, pyrrolidin-1-yl-(CH₂)ₘ, pyridinyl-(CH₂)ₘ, imidazolyl-(CH₂)ₘ, or imidazol-1-yl-(CH₂)ₘ; and
n and m are, respectively, integers from zero to two, inclusive, and from zero to four, inclusive.

54. The compound of claim 53, wherein R¹, R² and R³ are each hydrogen.

55. The compound of claim 53, wherein W is morpholin-4-yl-alkoxy.
